SEQUENCE LISTING (7 sequences)

SEQ ID NO: 1 - DNA, 596 bp, Bos taurus
tctgaagacctggatgcgggtggtaacggagcacgtgggtgttctcggagatcgacgatgtgccacgtgtggtttcttctgttttcaggccccagaagcccatcccgggaaggaaaatgcgctgtggacccctgtatcgattcctgtggctttggccctatctgtcttacgtggaggctgtgcccatctgcaaggtccaggatgacaccaaaaccctcatcaagacaattgtcaccaggatcaatgacatctcacacacggtagggagggactgggagacgaggtagaaccgtggccatcccgtgggggaccccagaggctggcggaggaggctgtgcagccttgcacagggccccagtggcctggacgcccccctggcataaagacagctcctctcctcctccacttcccttgcctcccgccttctcactctcctccctcccagaccggaatcctagtgcccaggcccagaaggagtcacagaggtcctggggtccccttggcaggtggccagaaccccagcagcagtccctctgggcctccatctcatttctagaatgttttagtcgttaggcattcttcctgcctggtaactg

SEQ ID NO: 2 - DNA, 596 bp, Bos taurus
tctgaagacctggatgcgggtggtaacggagcacgtgggtgttctcggagatcgacgatgtgccacgtgtggtttcttctgttttcaggccccagaagcccatcccgggaaggaaaatgcgctgtggacccctgtatcgattcctgtggctttggccctatctgtcttacgtggaggctgtgcccatccgcaaggtccaggatgacaccaaaaccctcatcaagacaattgtcaccaggatcaatgacatctcacacacggtagggagggactgggagacgaggtagaaccgtggccatcccgtgggggaccccagaggctggcggaggaggctgtgcagccttgcacagggccccagtggcctggacgcccccctggcataaagacagctcctctcctcctccacttcccttgcctcccgccttctcactctcctccctcccagaccggaatcctagtgcccaggcccagaaggagtcacagaggtcctggggtccccttggcaggtggccagaaccccagcagcagtccctctgggcctccatctcatttctagaatgttttagtcgttaggcattcttcctgcctggtaactg

SEQ ID NO: 3 - protein, 167 aa, Bos taurus
MRCGPLYRFLWLWPYLSYVEAVPIRKVQDDTKTLIKTIVTRINDISHTQSVSSKQRVTGLDFIPGLHPLLSLSKMDQTLAIYQQILTSLPSRNVVQISNDLENLRDLLHLLAASKSCPLPQVRALESLESLGVVLEASLYSTEVVALSRLQGSLQDMLRQLDLSPGC

SEQ ID NO: 4 - DNA, 20 bp, Artificial Sequence
  Description of Artificial Sequence Primer
agggatgcctggacacaaga

SEQ ID NO: 5 - DNA, 22 bp, Artificial Sequence
  Description of Artificial Sequence Primer
attgccaccaccagcagcacca

SEQ ID NO: 6 - DNA, 22 bp, Artificial Sequence
  Description of Artificial Sequence Primer
catctgctatgcgaatgctttg

SEQ ID NO: 7 - DNA, 20 bp, Artificial Sequence
  Description of Artificial Sequence Primer
gctaattatattgtaagaca

(19) United States
(12) Patent Application Publication
(10) Pub. No.: US 2005/0065736 A1
(43) Pub. Date: Mar. 24, 2005

(54) Systems and methods for improving efficiencies in livestock production

(76) Inventors: Stewart William Bauck, Cumming, GA (US); Michael Edward Hay Hutton, Montreal (CA); John Johnson, Alpharetta, GA (US); Eric Lloyd Marston, Atlanta, GA (US); Rodney D. Goodall, Cumming, GA (US)

Correspondence Address: Judy JARECKI-BLACK, Ph.D., J.D., 3239 Satellite Blvd., Duluth, GA, 30096, US

(21) Appl. No.: 10/891,256

(22) Filed: Jul. 13, 2004

Related U.S. Application Data
(60) Provisional application No. 60/487,784, filed on Jul. 15, 2003.
(60) Provisional application No. 60/560,115, filed on Apr. 7, 2004.
(60) Provisional application No. 60/509,775, filed on Oct. 8, 2003.

(51) Int. Cl. G06F 17/00; G01N 33/50; G01N 33/48; G06F 19/00

(52) U.S. Cl. 702/20; 700/90

(57) ABSTRACT

The present invention is directed to methods and systems for improving the efficiency of livestock production using genetic information obtained from the animal. The methods of the invention comprise obtaining a genetic sample from an animal or embryo, determining the genotype of the animal or embryo with respect to specific quality traits, grouping animals with like genotypes, and optionally, further sub-grouping animals based on like phenotypes. Based on the genotype, an animal is treated in a particular way. For example, uniform feeding regimens are designed for a particular group so as to maximize feed efficiencies and accurately predict slaughter times among like animals possessing a desired quality trait. Such methods include obtaining and maintaining the genetic data obtained from each animal, and optionally other data relating to the animal's health, condition or parentage, or to its herd, and providing this data to others through systems that are web-based, contained in a database, or attached to the animal itself such as by an implanted microchip.

### INCORPORATION BY REFERENCE

This Application claims priority to U.S. provisional Application Ser. No. 60/487,784 entitled: “Business Method for Improving Livestock production by Genotype” filed Jul. 15, 2003 now abandoned and to U.S. provisional Application Ser. No. 60/560,115 filed Apr. 7, 2004 entitled: “Business Method for Improving Livestock Production by Genotype”. All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and maybe employed in the practice of the invention. The disclosures of U.S. Provisional Application Ser. No. 60/466,523 entitled “METHOD FOR IMPROVING EFFICIENCIES IN LIVESTOCK PRODUCTION”, filed Apr. 29, 2003, U.S. Provisional Application Ser. No. 60/509,775 entitled “METHOD FOR IMPROVING FEED CONVERSION EFFICIENCY IN LIVESTOCK PRODUCTION”, filed Oct. 8, 2003, and U.S. application Ser. No. 10/770,307 entitled “METHODS FOR IDENTIFYING AND MANAGING LIVESTOCK BY GENOTYPE”, filed Feb. 2, 2004 are hereby incorporated by reference in their entireties.

### FIELD OF THE INVENTION

The present invention relates to methods and systems of identification and management of livestock. More specifically, the invention relates to methods and systems, including network-based processes, to manage data such as identification and management of livestock in groups which, based on genotyping, have predictable quality traits.

### BACKGROUND OF THE INVENTION

Animals account for almost 20 percent of the world's food consumption. It follows, then, that animal-based food products are a major source of revenue throughout the world. In the United States alone, beef production is the fourth largest manufacturing industry and accounts for nearly 25 percent of the farm sector cash receipts and seven percent of supermarket sales each year.

Body condition of the animals is a determinant of market readiness in commercial livestock feeding and finishing operations. The term “body condition” is used in the livestock industry in reference to the state of development of a livestock animal that is a function of frame type or size, and the amount of intramuscular fat and back fat exhibited by an animal. It is typically determined subjectively and through experienced visual appraisal of live animals. The fat deposition, or the amount of intramuscular fat and back fat on an animal carcass, is important to industry participants because carcasses exhibiting desired amounts and proportions of such fats can often be sold for higher prices than carcasses that exhibit divergences from such desired amounts and proportions. Furthermore, the desired carcass fat deposition often varies among different markets and buyers, and it also often varies with time within single markets and among particular buyers in response to public demand trends with respect to desired fat and marbling in meats.

Presently, cattle entering a feed lot are divided into groups according to estimated age, frame size, breed, weight, and so forth. By making such a division, the feed lot owner is attempting to group the cattle so that the group can be penned together and fed the same ration and will be ready for slaughter at the same time. Weight and visual clues are the only means possible to sort cattle for feed lot grouping. Therefore, there exists a need in the industry for objectively grouping livestock entering a feed lot.

Similarly, animals used for dairy or eggs are priced according to production expectations. The greater the production expectations, the greater the price realized by the feed operator. Regardless of the particular market preference at a given time, the feed lot operator will be trying to tailor his animals to meet some similar standard that will cause a meat packer or commercial purchaser to pay the highest price in accordance with currently prevailing market preferences.

The feed lot operator's costs include operations costs for the lot, such as labor, capital, maintenance, and the like, plus the cost of feeding the animals. While the cost of acquiring each animal in a group can vary somewhat, the feed lot operator's costs are the same for each animal in the group since they are fed the same amount of feed and occupy space in the feed lot for the same amount of time. Thus, the price reductions for carcasses falling outside the desirable range fall directly to the feed lot operator's bottom line, resulting in reduced profits.

One way for the feed lot operator to reduce costs and increase profits is to alter the time an animal spends on the lot, thus reducing the feed costs. Longer residence times are usually only profitable if the result is an animal with a more profitable grade. Because the residence time and the feed schedule are based on visual inspections, there is no objective way to predict which animals will result in higher grades of meat. Similarly, meat packers predict the carcass grade based on visual inspection of the live animals, and after slaughter, based on a series of fat measurements. It is common that the post-slaughter inventory of a specific, desired grade of meat does not meet with a packers pre-slaughter demand. This results in an uncertain inventory, and often means that the packer must purchase additional animals to fill a desired inventory. Accordingly, there exists a need in the art for accurately and objectively predicting animal meat grades in advance of slaughter. Additionally, methods and systems are needed for tracking individual animals easily and accurately and maintaining data associated with individual animals.

Additionally, other considerations important for animal management practices include treatment for and prevention of infectious diseases. Current animal production practice involves treating animals en masse for infectious diseases, either prophylactically or when clinical signs arise, because individual animals at risk can not be easily identified. Thus, a method that allows producers to identify individual animals at risk for infection is highly desirable and would save time and money for the producers.

Also desirable is a method that allows for the compilation, retrieval, and sharing of genetic information in a database such that genetic predisposition to disease, health conditions, and other phenotypic traits of economic importance, is available upon accessing the database. Such data could be available from a central database or coded, for example, on a chip implanted in the individual animal.

Because of these deficiencies and others inherent in the prior art, it would be advantageous to provide a business method that provides for increased production efficiencies in livestock animals, including cattle, swine, sheep and the like.

### SUMMARY OF THE INVENTION

Citation or identification of any document in this application is not an admission that such document is available as prior art to the present invention.

The present invention is directed to computer-assisted methods and systems for improving the efficiency of livestock production using genetic information obtained from the animal. More particularly, methods of the invention comprise obtaining a genetic sample from each animal in a herd of livestock, determining the genotype of each animal with respect to specific quality traits, grouping animals with like genotypes, and optionally, further sub-grouping animals based on like phenotypes. Methods of the invention include obtaining and maintaining the genetic data obtained from each animal, and optionally other data relating to the animal's health, condition or parentage, or to its herd, and providing this data to others through systems that are web-based, contained in a database, or attached to the animal itself such as by implanted microchip.

In one embodiment of the present invention, the homozygosity or heterozygosity of each animal is determined with respect to alleles of one or more genes encoding a trait-specific polypeptide or a control function, or other identifying nucleic acid sequences. Animals determined to be homozygous for a particular trait, either homozygous recessive or homozygous dominant, are grouped with other like-homozygous animals and are segregated from animals determined to be either the other homozygous or heterozygous for that trait. Each group is fed according to a regimen and for a period of time designed to optimize the desired trait in the animal while efficiently managing the resources of the livestock producer.

In another embodiment of the present invention, once the homozygosity or heterozygosity is determined and the animals are segregated, animals are bred within each group or subgroup. The resulting animals allow breeders to more accurately predict the natural genetic potential to produce quality traits. The offspring are then, themselves, genotyped, segregated with like-type animals and fed according to a regimen and for a period of time designed to optimize the desired trait in the animal product while efficiently managing the resources of the livestock producer.

An advantageous aspect of the present invention is directed to a computer system and computer-assisted method for predicting quality traits for livestock possessing specific genetic predispositions. In this method, an animal is genotyped based on specific genetic traits, segregated with like-genotype animals, and bred or fed according to regimens specific to optimize the trait or characteristic. In one embodiment, a farmer can predict which animals will possess certain quality traits based on breeding of like-type animals. In another embodiment, a farmer can maximize feeding regimen efficiencies based on genetic predispositions. In a further embodiment, a meat packer or other commercial purchaser can base his purchase of livestock on the results of genotyping.

The present invention provides computer-assisted methods for genotyping animals, collecting and storing the data resulting from genotyping, classifying livestock based on the genetic data, and formulating feed and slaughter schedules for livestock possessing like genetic traits. The methods of the present invention optimize the efficiencies of raising livestock since the producer or packer can predict optimum feed quantities and slaughter schedules for each animal, based on the animal's predisposition to provide a desired product characteristic.

The present invention comprises computer-assisted methods and systems for acquiring genetic data, particularly data related to quality traits of the breed of animal and associating that data with other data about the animal or its herd, and maintaining that data in ways that are accessible.

One embodiment of the present invention is to provide a computer system and method for improving efficiencies in livestock production. Such method comprises genotyping animals based on one or more genetic traits, storing the genetic data in a database, accessing the database to identify common traits among animals and grouping the animals. The invention also provides for the input of other data, such as health and/or vaccination records, into the database. Alternatively the present invention provides for linking specific genotypes with a predisposition to medical conditions including propensity of an individual animal to infectious agents; susceptibility of that animal to specific medical treatment; genetic predisposition to an undesirable phenotypic trait; and the like. In another advantageous embodiment, the database can be linked to other databases.

An embodiment of the present invention is to provide a method for breeding animals to meet particular body characteristics. The method provides that each animal is genotyped to determine the absence or presence of a specific trait, then the animal is segregated into groups based on like genotype, and optionally, based on other like-physical characteristics. Breeding occurs between animals within the same group and having like characteristics and the offspring are then genotyped and fed according to an optimized feed and slaughter schedule.

A further embodiment of the present invention is to provide a computer-assisted method and system for managing livestock feeding regimens comprising genotyping animals based on one or more genetic traits, storing the genetic data in a database, accessing the database to identify common traits among animals, grouping animals having the greatest genetic predisposition to produce a specific quality trait with animals having like predisposition, and feeding the group a diet conducive to produce such trait for a period of time which optimizes the cost of feeding. In one embodiment, animals having a high genetic predisposition to produce lean meat are grouped together and fed a specific diet, and for a specific period of time, such that the feeding regimen is conducive to producing lean meat.

Another embodiment of the present invention is to provide a computer system and method for managing animals entering a feed lot comprising genotyping animals based on one or more genetic traits, storing the genetic data in a database, accessing the database to identify common traits among animals, grouping the animals based on like genotypes, and optionally based on like phenotypes, and feeding the group according to the most efficient feeding regimen for said genotype.

Yet another embodiment of the present invention is to provide a computer system and method for predicting the quality of meat obtained from a herd of animals by genotyping animals based on one or more genetic traits, storing the genetic data in a database, accessing the database to identify common traits among animals, grouping the animals according to like genotypes, and optionally like phenotypes, and breeding animals with like genotypes, and optionally, feeding the group according to the most efficient feeding regimen for said genotype. In one embodiment, packers can respond to market signals for specific types of meat by purchasing animals having greater or lesser genetic predisposition to lay down fat, metabolize energy, produce milk, lay eggs, or other physical traits.

A further embodiment of the present invention provides a livestock management method for designing and implementing treatment for, and prevention of, infectious diseases. In contrast to current animal production practice involving the treatment of animals en masse for infectious diseases, either prophylactically or when clinical signs arise in a herd, the present invention provides a method to identify individual animals at risk for infection using genetic markers. In one embodiment the genetic information of an entire herd is stored in a database and may be accessed to determine treatment parameters such as identifying animals susceptible to certain infectious agents based on the individual's genetic propensity.

Also provided by the present invention is a method that allows for the compilation, retrieval, and sharing of genetic information in a database such that genetic predispositon to disease, health conditions, and other phenotypic traits of economic importance is available upon accessing the database. Such data could be available from a central database or coded, for example, on a chip implanted in the individual animal.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIG. 1 shows a flowchart illustrating the general overview of input, intermediate steps, and output of the method according to the present invention.

### DETAILED DESCRIPTION

In the description that follows, a number of terms are extensively utilized. In order to provide a clear and consistent understanding of the specification and claims, including the scope to be given such terms, the following terminology is provided:

An “amplification primer” is an oligonucleotide that is capable of annealing adjacent to a target sequence and serving as an initiation point for DNA synthesis when placed under conditions in which synthesis of a primer extension product which is complementary to a nucleic acid strand is initiated.

By “amplifying a segment” as used herein, is meant the production of sufficient multiple copies of the segment to permit relatively facile manipulation of the segment. Manipulation refers to both physical and chemical manipulation, that is, the ability to move bulk quantities of the segment around and to conduct chemical reactions with the segment that result in detectable products. A “segment” of a polynucleotide refers to an oligonucleotide that is a partial sequence of entire nucleotide sequence of the polynucleotide. A “modified segment” refers to a segment in which one or more natural nucleotides have been replaced with one or more modified nucleotides. A “modified, labeled segment refers to a modified segment that also contains a nucleotide, which is different from the modified nucleotide or nucleotides therein, and which is detectably labeled.

By “analysis” is meant either detection of variance in the nucleotide sequence among two or more related polynucleotides or, in the alternative, the determination of the full nucleotide sequence of a polynucleotide. By “analyzing” the hybridized fragments for an incorporated detectable label identifying the suspected polymorphism is meant that, at some stage of the sequence of events that leads to hybridized fragments, a label is incorporated. The label may be incorporated at virtually any stage of the sequence of events including the amplification, the cleavage or the hybridization procedures. The label may even be introduced into the sequence of events after cleavage but before hybridization or even after hybridization. The label so incorporated is then observed visually or by instrumental means. The presence of the label identifies the polymorphism due to the fact that the fragments obtained during cleavage are specific to the modified nucleotide(s) used in the amplification and at least one of the modified nucleotide is selected so as to replace a nucleotide involved in the polymorphism.

The term “animal” is used herein to include all vertebrate animals, including humans. It also includes an individual animal in all stages of development, including embryonic and fetal stages. As used herein, the term “production animals” is used interchangeably with “livestock animals” and refers generally to animals raised primarily for food. For example, such animals include, but are not limited to, cattle (bovine), sheep (ovine), pigs (porcine or swine), poultry (avian), and the like. As used herein, the term “cow” or “cattle” is used generally to refer to an animal of bovine origin of any age. Interchangeable terms include “bovine”, “calf”, “steer”, “bull”, “heifer” and the like. As used herein, the term “pig” or “swine” is used generally to refer to an animal of porcine origin of any age. Interchangeable terms include “piglet”, “sow” and the like.

The term “antisense” is intended to refer to polynucleotide molecules complementary to a portion of an RNA marker of the ob gene, as defined herein. “Complementary” polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

By the term “complementarity” or “complementary” is meant, for the purposes of the specification or claims, a sufficient number in the oligonucleotide of complementary base pairs in its sequence to interact specifically (hybridize) with the target nucleic acid sequence of the gene polymorphism to be amplified or detected. As known to those skilled in the art, a very high degree of complementarity is needed for specificity and sensitivity involving hybridization, although it need not be 100%. Thus, for example, an oligonucleotide that is identical in nucleotide sequence to an oligonucleotide disclosed herein, except for one base change or substitution, may function equivalently to the disclosed oligonucleotides. A “complementary DNA” or “cDNA” gene includes recombinant genes synthesized by reverse transcription of messenger RNA (“mRNA”).

By the term “composition” is meant, for the purposes of the specification or claims, a combination of elements which may include one or more of the following: the reaction buffer for the respective method of enzymatic amplification, plus one or more oligonucleotides specific for gene polymorphisms, wherein said oligonucleotide is labeled with a detectable moiety.

By the terms “consisting essentially of a nucleotide sequence” is meant, for the purposes of the specification or claims, the nucleotide sequence disclosed, and also encompasses nucleotide sequences which are identical except for a one base change or substitution therein.

A “cyclic polymerase-mediated reaction” refers to a biochemical reaction in which a template molecule or a population of template molecules is periodically and repeatedly copied to create a complementary template molecule or complementary template molecules, thereby increasing the number of the template molecules over time.

“Denaturation” of a template molecule refers to the unfolding or other alteration of the structure of a template so as to make the template accessible to duplication. In the case of DNA, “denaturation” refers to the separation of the two complementary strands of the double helix, thereby creating two complementary, single stranded template molecules. “Denaturation” can be accomplished in any of a variety of ways, including by heat or by treatment of the DNA with a base or other denaturant.

A “detectable amount of product” refers to an amount of amplified nucleic acid that can be detected using standard laboratory tools. A “detectable marker” refers to a nucleotide analog that allows detection using visual or other means. For example, fluorescently labeled nucleotides can be incorporated into a nucleic acid during one or more steps of a cyclic polymerase-mediated reaction, thereby allowing the detection of the product of the reaction using, e.g. fluorescence microscopy or other fluorescence-detection instrumentation.

By the term “detectable moiety” is meant, for the purposes of the specification or claims, a label molecule (isotopic or non-isotopic) which is incorporated indirectly or directly into an oligonucleotide, wherein the label molecule facilitates the detection of the oligonucleotide in which it is incorporated when the oligonucleotide is hybridized to amplified gene polymorphisms sequences. Thus, “detectable moiety” is used synonymously with “label molecule”. Synthesis of oligonucleotides can be accomplished by any one of several methods known to those skilled in the art. Label molecules, known to those skilled in the art as being useful for detection, include chemiluminescent or fluorescent molecules. Various fluorescent molecules are known in the art which are suitable for use to label a nucleic acid substrate for the method of the present invention. The protocol for such incorporation may vary depending upon the fluorescent molecule used. Such protocols are known in the art for the respective fluorescent molecule.

By “detectably labeled” is meant that a fragment or an oligonucleotide contains a nucleotide that is radioactive, that is substituted with a fluorophore or some other molecular species that elicits a physical or chemical response can be observed by the naked eye or by means of instrumentation such as, without limitation, scintillation counters, calorimeters, UV spectrophotometers and the like. As used herein, a “label” or “tag” refers to a molecule that, when appended by, for example, without limitation, covalent bonding or hybridization, to another molecule, for example, also without limitation, a polynucleotide or polynucleotide fragment, provides or enhances a means of detecting the other molecule. A fluorescence or fluorescent label or tag emits detectable light at a particular wavelength when excited at a different wavelength. A radiolabel or radioactive tag emits radioactive particles detectable with an instrument such as, without limitation, a scintillation counter. Other signal generation detection methods include: chemiluminescence, electrochemiluminescence, raman, colorimetric, hybridization protection assay, and mass spectrometry.

“DNA amplification” as used herein refers to any process that increases the number of copies of a specific DNA sequence by enzymatically amplifying the nucleic acid sequence. A variety of processes are known. One of the most commonly used is the polymerase chain reaction (PCR) process of Mullis as described in U.S. Pat. Nos. 4,683,195 and 4,683,202. PCR involves the use of a thermostable DNA polymerase, known sequences as primers, and heating cycles, which separate the replicating deoxyribonucleic acid (DNA), strands and exponentially amplify a gene of interest. Any type of PCR, such as quantitative PCR, RT-PCR, hot start PCR, LAPCR, multiplex PCR, touchdown PCR, etc., may be used. Advantageously, real-time PCR is used. In general, the PCR amplification process involves an enzymatic chain reaction for preparing exponential quantities of a specific nucleic acid sequence. It requires a small amount of a sequence to initiate the chain reaction and oligonucleotide primers that will hybridize to the sequence. In PCR the primers are annealed to denatured nucleic acid followed by extension with an inducing agent (enzyme) and nucleotides. This results in newly synthesized extension products. Since these newly synthesized sequences become templates for the primers, repeated cycles of denaturing, primer annealing, and extension results in exponential accumulation of the specific sequence being amplified. The extension product of the chain reaction will be a discrete nucleic acid duplex with a termini corresponding to the ends of the specific primers employed.

“DNA” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

By the terms “enzymatically amplify” or “amplify” is meant, for the purposes of the specification or claims, DNA amplification, i.e., a process by which nucleic acid sequences are amplified in number. There are several means for enzymatically amplifying nucleic acid sequences. Currently the most commonly used method is the polymerase chain reaction (PCR). Other amplification methods include LCR (ligase chain reaction) which utilizes DNA ligase, and a probe consisting of two halves of a DNA segment that is complementary to the sequence of the DNA to be amplified, enzyme QB replicase and a ribonucleic acid (RNA) sequence template attached to a probe complementary to the DNA to be copied which is used to make a DNA template for exponential production of complementary RNA; strand displacement amplification (SDA); QB replicase amplification (QBRA); self-sustained replication (3SR); and NASBA (nucleic acid sequence-based amplification), which can be performed on RNA or DNA as the nucleic acid sequence to be amplified.

The “extension of the primer molecules” refers to the addition of nucleotides to a primer molecule so as to synthesize a nucleic acid complementary to a template molecule. “Extension of the primer molecules” does not necessarily imply that the primer molecule is extended to synthesize a complete complementary template molecule. Rather, even if only a fraction of the template molecule has been copied, the primer is still considered extended.

A “fragment” of a molecule such as a protein or nucleic acid is meant to refer to any portion of the amino acid or nucleotide genetic sequence.

As used herein, “fluorescence resonance energy transfer pair” or “FRET pair” refers to a pair of fluorophores comprising a donor fluorophore and acceptor fluorophore, wherein the donor fluorophore is capable of transferring resonance energy to the acceptor fluorophore. In other words the emission spectrum of the donor fluorophore overlaps the absorption spectrum of the acceptor fluorophore. In advantageous fluorescence resonance energy transfer pairs, the absorption spectrum of the donor fluorophore does not substantially overlap the absorption spectrum of the acceptor fluorophore. As used herein, “a donor oligonucleotide probe” refers to an oligonucleotide that is labeled with a donor fluorophore of a fluorescent resonance energy transfer pair. As used herein, “an acceptor oligonucleotide probe” refers to an oligonucleotide that is labeled with an acceptor fluorophore of a fluorescent resonance energy transfer pair. As used herein, “FRET oligonucleotide pair” refers to the donor oligonucleotide probe and the acceptor oligonucleotide probe pair that form a fluorescence resonance energy transfer relationship when the donor oligonucleotide probe and the acceptor oligonucleotide probe are both hybridized to their complementary target nucleic acid sequences. Two separate FRET oligonucleotide pairs, each specific for one locus and each comprising a different acceptor dye may be used at the same time. Acceptable fluorophore pairs for use as fluorescent resonance energy transfer pairs are well know to those skilled in the art and include, but are not limited to, fluorescein/rhodamine, phycoerythrin/Cy7, fluorescein/Cy5, fluorescein/Cy5.5, fluorescein/LC Red 640, and fluorescein/LC Red 705.

A “functional derivative” of a sequence, either protein or nucleic acid, is a molecule that possesses a biological activity (either functional or structural) that is substantially similar to a biological activity of the protein or nucleic acid sequence. A functional derivative of a protein may or may not contain post-translational modifications such as covalently linked carbohydrate, depending on the necessity of such modifications for the performance of a specific function. The term “functional derivative” is intended to include the “fragments,” “segments,” “variants,” “analogs,” or “chemical derivatives” of a molecule.

As used herein, the term “genome” refers to all the genetic material in the chromosomes of a particular organism. Its size is generally given as its total number of base pairs. Within the genome, the term “gene” refers to an ordered sequence of nucleotides located in a particular position on a particular chromosome that encodes specific functional product (e.g., a protein or RNA molecule). For example, it is known that the protein leptin is encoded by the ob (obese) gene and appears to be involved in the regulation of appetite, basal metabolism and fat deposition In general, an animal's genetic characteristics, as defined by the nucleotide sequence of its genome, are known as its “genotype,” while the animal's physical traits are described as its “phenotype.”

By “heterozygous” or “heterozygous polymorphism” is meant that the two alleles of a diploid cell or organism at a given locus are different, that is, that they have a different nucleotide exchanged for the same nucleotide at the same place in their sequences.

By “homozygous” it is meant that the two alleles of a diploid cell or organism at a given locus are identical, that is, that they have the same nucleotide for nucleotide exchange at the same place in their sequences.

By “hybridization” or “hybridizing,” as used herein, is meant the formation of A-T and C-G base pairs between the nucleotide sequence of a fragment of a segment of a polynucleotide and a complementary nucleotide sequence of an oligonucleotide. By complementary is meant that at the locus of each A, C, G or T (or U in a ribonucleotide) in the fragment sequence, the oligonucleotide sequenced has a T, G, C or A, respectively. The hybridized fragment/oligonucleotide is called a “duplex.”

A “hybridization complex”, such as in a sandwich assay, means a complex of nucleic acid molecules including at least the target nucleic acid and sensor probe. It may also include an anchor probe.

By “immobilized on a solid support” is meant that a fragment, primer or oligonucleotide is attached to a substance at a particular location in such a manner that the system containing the immobilized fragment, primer or oligonucleotide may be subjected to washing or other physical or chemical manipulation without being dislodged from that location. A number of solid supports and means of immobilizing nucleotide-containing molecules to them are known in the art; any of these supports and means may be used in the methods of this invention.

As used herein, the term “increased weight gain” means a biologically significant increase in weight gain above the mean of a given population.

As used herein, the term “locus” or “loci” refers to the site of a gene on a chromosome. Pairs of genes, known as “alleles” control the hereditary trait produced by a gene locus. Each animal's particular combination of alleles is referred to as its “genotype”. Where both alleles are identical the individual is said to be homozygous for the trait controlled by that gene pair; where the alleles are different, the individual is said to be heterozygous for the trait.

“Matrix-assisted laser desorption/ionisation-time of flight mass spectrometry” (“MALDI-TOF MS”) refers to a technique in which a co-precipitate of an UV-light absorbing matrix and a biomolecule is irradiated by a nanosecond laser pulse. Most of the laser energy is absorbed by the matrix, which prevents unwanted fragmentation of the biomolecule. The ionized biomolecules are accelerated in an electric field and enter the flight tube. During the flight in this tube, different molecules are separated according to their mass to charge ratio and reach the detector at different times. In this way each molecule yields a distinct signal. The method is used for detection and characterization of biomolecules, such as proteins, peptides, oligosaccharides and oligonucleotides, with molecular masses between 400 and 350,000 Da. It is a very sensitive method, which allows the detection of low (10⁻¹⁵ to 10⁻¹⁸ mole) quantities of sample with an accuracy of about 0.1 to about 0.01%.

A “melting temperature” is meant the temperature at which hybridized duplexes dehybridize and return to their single-stranded state. Likewise, hybridization will not occur in the first place between two oligonucleotides, or, herein, an oligonucleotide and a fragment, at temperatures above the melting temperature of the resulting duplex. It is presently advantageous that the difference in melting point temperatures of oligonucleotide-fragment duplexes of this invention be from about 1° C. to about 10° C. so as to be readily detectable.

As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but advantageously is double-stranded DNA. An “isolated” nucleic acid molecule is one that is separated from other nucleic acid molecules that are present in the natural source of the nucleic acid. A “nucleoside” refers to a base linked to a sugar. The base may be adenine (A), guanine (G) (or its substitute, inosine (I)), cytosine (C), or thymine (T) (or its substitute, uracil (U)). The sugar may be ribose (the sugar of a natural nucleotide in RNA) or 2-deoxyribose (the sugar of a natural nucleotide in DNA). A “nucleotide” refers to a nucleoside linked to a single phosphate group.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides may be chemically synthesized and may be used as primers or probes. Oligonucleotide means any nucleotide of more than 3 bases in length used to facilitate detection or identification of a target nucleic acid, including probes and primers.

“Polymerase chain reaction” or “PCR” refers to a thermocyclic, polymerase-mediated, DNA amplification reaction. A PCR typically includes template molecules, oligonucleotide primers complementary to each strand of the template molecules, a thermostable DNA polymerase, and deoxyribonucleotides, and involves three distinct processes that are multiply repeated to effect the amplification of the original nucleic acid. The three processes (denaturation, hybridization, and primer extension) are often performed at distinct temperatures, and in distinct temporal steps. In many embodiments, however, the hybridization and primer extension processes can be performed concurrently. The nucleotide sample to be analyzed may be PCR amplification products provided using the rapid cycling techniques described in U.S. Pat. Nos. 6,569,672; 6,569,627; 6,562,298; 6,556,940; 6,569,672; 6,569,627; 6,562,298; 6,556,940; 6,489,112; 6,482,615; 6,472,156; 6,413,766; 6,387,621; 6,300,124; 6,270,723; 6,245,514; 6,232,079; 6,228,634; 6,218,193; 6,210,882; 6,197,520; 6,174,670; 6,132,996; 6,126,899; 6,124,138; 6,074,868; 6,036,923; 5,985,651; 5,958,763; 5,942,432; 5,935,522; 5,897,842; 5,882,918; 5,840,573; 5,795,784; 5,795,547; 5,785,926; 5,783,439; 5,736,106; 5,720,923; 5,720,406; 5,675,700; 5,616,301; 5,576,218 and 5,455,175, the disclosures of which are incorporated by reference in their entireties. Other methods of amplification include, without limitation, NASBR, SDA, 3SR, TSA and rolling circle replication. It is understood that, in any method for producing a polynucleotide containing given modified nucleotides, one or several polymerases or amplification methods may be used. The selection of optimal polymerization conditions depends on the application.

A “polymerase” is an enzyme that catalyzes the sequential addition of monomeric units to a polymeric chain, or links two or more monomeric units to initiate a polymeric chain. In advantageous embodiments of this invention, the “polymerase” will work by adding monomeric units whose identity is determined by and which is complementary to a template molecule of a specific sequence. For example, DNA polymerases such as DNA pol 1 and Taq polymerase add deoxyribonucleotides to the 3′ end of a polynucleotide chain in a template-dependent manner, thereby synthesizing a nucleic acid that is complementary to the template molecule. Polymerases may be used either to extend a primer once or repetitively or to amplify a polynucleotide by repetitive priming of two complementary strands using two primers.

A “polynucleotide” refers to a linear chain of nucleotides connected by a phosphodiester linkage between the 3′-hydroxyl group of one nucleoside and the 5′-hydroxyl group of a second nucleoside which in turn is linked through its 3′-hydroxyl group to the 5′-hydroxyl group of a third nucleoside and so on to form a polymer comprised of nucleosides liked by a phosphodiester backbone. A “modified polynucleotide” refers to a polynucleotide in which one or more natural nucleotides have been partially or substantially completely replaced with modified nucleotides.

A “primer” is a short oligonucleotide, the sequence of which is complementary to a segment of the template which is being replicated, and which the polymerase uses as the starting point for the replication process. By “complementary” is meant that the nucleotide sequence of a primer is such that the primer can form a stable hydrogen bond complex with the template; i.e., the primer can hybridize to the template by virtue of the formation of base-pairs over a length of at least ten consecutive base pairs.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

“Probes” refer to nucleic acid sequences of variable length, used in the detection of identical, similar, or complementary nucleic acid sequences by hybridization. An oligonucleotide sequence used as a detection probe may be labeled with a detectable moiety. Various labeling moieties are known in the art. Said moiety may, for example, either be a radioactive compound, a detectable enzyme (e.g. horse radish peroxidase (HRP)) or any other moiety capable of generating a detectable signal such as a calorimetric, fluorescent, chemiluminescent or electrochemiluminescent signal. The detectable moiety may be detected using known methods. In one embodiment the probe oligomers are generally 8 to 44-mers and advantageously about 10 to 12-mers and advantageously about 11-mers.

As used herein, the term “protein” refers to a large molecule composed of one or more chains of amino acids in a specific order. The order is determined by the base sequence of nucleotides in the gene coding for the protein. Proteins are required for the structure, function, and regulation of the body's cells, tissues, and organs. Each protein has a unique function.

As used herein, the terms “quality traits” or “physical characteristics” or “phenotypes” refer to advantageous properties of the animal resulting from genetics. Quality traits include, but are not limited to, the animal's genetic ability to efficiently metabolize energy, produce meat or milk, put on intramuscular fat, lay eggs, produce offspring, produce particular proteins in meat or milk, retain protein in milk, or resist disease. Physical characteristics include marbled or lean meats. The terms are used interchangeably.

A “restriction enzyme” refers to an endonuclease (an enzyme that cleaves phosphodiester bonds within a polynucleotide chain) that cleaves DNA in response to a recognition site on the DNA. The recognition site (restriction site) consists of a specific sequence of nucleotides typically about 4-8 nucleotides long.

A “single nucleotide polymorphism” or “SNP” refers to polynucleotide that differs from another polynucleotide by a single nucleotide exchange. For example, without limitation, exchanging one A for one C, G or T in the entire sequence of polynucleotide constitutes a SNP. Of course, it is possible to have more than one SNP in a particular polynucleotide. For example, at one locus in a polynucleotide, a C may be exchanged for a T, at another locus a G may be exchanged for an A and so on. When referring to SNPS, the polynucleotide is most often DNA and the SNP is one that usually results in a deleterious change in the genotype of the organism in which the SNP occurs.

As used herein, a “template” refers to a target polynucleotide strand, for example, without limitation, an unmodified naturally-occurring DNA strand, which a polymerase uses as a means of recognizing which nucleotide it should next incorporate into a growing strand to polymerize the complement of the naturally-occurring strand. Such DNA strand may be single-stranded or it may be part of a double-stranded DNA template. In applications of the present invention requiring repeated cycles of polymerization, e.g., the polymerase chain reaction (PCR), the template strand itself may become modified by incorporation of modified nucleotides, yet still serve as a template for a polymerase to synthesize additional polynucleotides.

A “thermocyclic reaction” is a multi-step reaction wherein at least two steps are accomplished by changing the temperature of the reaction.

A “thermostable polymerase” refers to a DNA or RNA polymerase enzyme that can withstand extremely high temperatures, such as those approaching 100° C. Often, thermostable polymerases are derived from organisms that live in extreme temperatures, such as Thermus aquaticus. Examples of thermostable polymerases include Taq, Tth, Pfu, Vent, deep vent, UlTma, and variations and derivatives thereof.

A “variance” is a difference in the nucleotide sequence among related polynucleotides. The difference may be the deletion of one or more nucleotides from the sequence of one polynucleotide compared to the sequence of a related polynucleotide, the addition of one or more nucleotides or the substitution of one nucleotide for another. The terms “mutation,” “polymorphism” and “variance” are used interchangeably herein. As used herein, the term “variance” in the singular is to be construed to include multiple variances; i.e., two or more nucleotide additions, deletions and/or substitutions in the same polynucleotide. A “point mutation” refers to a single substitution of one nucleotide for another.

The present invention differs from current practice by using genetic test results to characterize animals. Rather than rely on a growth curve, ultrasound examination or visual inspection of animal traits, the present invention allows the farmer to feed livestock according to the individual animal's genetic traits. According to the method of the present invention, it is possible to select a desired trait, such as fat content, identify the polypeptide which specifically encodes for a gene associate with that trait, and genotype animals possessing the associated gene.

In the example of fat content, it is known that leptin, a 16-kDa adipocyte-specific polypeptide, is encoded by the ob (obese) gene and appears to be involved in the regulation of appetite, basal metabolism and fat deposition. The ob gene has been mapped to specific chromosomes in several different animals, allowing the gene to be sequenced in several different species. In the case of leptin, there is significant conservation among the sequences of ob DNAs and leptin polypeptides from the tested species. Mutations in the coding sequences of the ob gene causing alterations in the amino acid sequence of the leptin polypeptide have been associated with hyperphagia, hypometabolic activity, and excessive fat deposition, i.e., a phenotype characterized by larger body size (a fat phenotype). In the method of the present invention, it is possible to identify the absence or presence of a specific ob allele, thus predicting which animals will or will not possess certain carcass characteristics, e.g., increased fat deposition, increased mean fat deposition, increased percent rib fat, and decreased percent rib lean. For the ob gene, the presence of 138-bp allele was positively associated with these characteristics. Thus, bulls homogenous for the 138-bp allele exhibited greater average fat deposition than heterozygous animals.

The present invention provides methods wherein the genetic information obtained from individual animals is cross-matched against markers known in the art to predict specific characteristics. Keeping with the fat content example, it is known that a cytosine (C) to thymine (T) transition within an exon (exon 2) of the ob gene correspond to an arginine (ARG) to cysteine (CYS) substitution in the leptin polypeptide. The exon 2 polymorphism is a C/T substitution located at position 305 of exon 2 of the bovine leptine gene according to &50365 (see, e.g., Buchanan et al. (2002) Genet Sel Evol. 34(1):105-16). Thus, it is known that the presence of a T-containing allele in bulls is predictive of fatter carcasses while bulls with a C-containing allele are known to be leaner. Thus, once the genotype of the animal is determined, it is evaluated to determine whether each individual animal possesses the desired trait, i.e., possesses the specific gene. Animals having like genotypes for a specific gene/characteristic are then grouped together. These like-genotype groupings serve as the basis for breeding, feeding and determining slaughter time. Accordingly, the like-genotype groupings provide a more objective method for determining mates for breeding, diets and lengths of feed cycles, and slaughter times.

The individual genotype data of each animal can be recorded and associated with various other data of the animal, e.g. health information, parentage, vaccination history, herd records, and the like. Such information can be forwarded to a government agency to provide traceability of a meat product, or it may serve as the basis for breeding, feeding and marketing information. Once the genotype data is established, and that data may or may not be associated with other data, the data is stored in an accessible database, such as a computer database or a microchip implanted in the animal.

Genetic tendencies can be predicted by the results of genotyping. A method and system of the invention comprises tissue sampling, extraction of genetic material from the sampled tissue, molecular genetic analysis of the genetic material, and where the tissue sample is taken from a meat product, comparison of the genotype with known animal genotypes stored on a database. It is contemplated by the methods and systems described herein that the continuity and integrity of each sample is maintained so that the data is accurate and reliable. Steps necessary for ensuring that the data is accurate and reliable are included in the methods and systems taught herein.

Additionally, the method of the present invention contemplates grouping animals according to their genotype in addition to using the phenotype criteria currently employed in feeding, breeding or growing stages practices. For example, in one embodiment of the present invention, feedlot operators who currently group livestock according to size and frame structures, among other phenotypic traits, would use the data obtained from animals' genotypes which correspond to an animal's propensity to exhibit a characteristic associated with the particular gene, and optionally any other associated data, in order to more efficiently manage production. Thus, the feeder is presented with opportunities for considerable efficiencies in livestock production.

Presently, the feeder feeds all his cattle the same, incurring the same costs for each animal, and typically, with excellent management practices, perhaps 40% will receive an optimal grade of Choice or Prime, and receive the premium price for the quality grade. Of these, a significant number will have excess fat and will thus receive a reduced yield grade. The balance of the cattle, 60%, will grade less than Choice or Prime, and thus receive a reduced price, although the feed lot costs incurred by the feeder are substantially the same for these cattle receiving the lesser grade. Grouping and feeding the cattle by genotype allows the feeder to treat each group differently with a view to optimizing management strategies and increasing profits.

A tissue sample may be taken from an animal at any time in the lifetime of an animal but before the carcass identity is lost. The tissue sample can comprise hair, including roots, hide, bone, buccal swabs, blood, saliva, milk, semen, embryos, muscle or any internal organs.

The tissue sample is marked with an identifying number or other indicia that relates the sample to the individual animal from which the sample was taken. The identity of the sample advantageously remains constant throughout the methods and systems of the invention thereby guaranteeing the integrity and continuity of the sample during extraction and analysis. Alternatively, the indicia may be changed in a regular fashion that ensures that the data, and any other associated data, can be related back to the animal from which the data was obtained.

The amount/size of sample required is known to those skilled in the art and for example, can be determined by the subsequent steps used in the method and system of the invention and the specific methods of analysis used. Ideally, the size/volume of the tissue sample retrieved should be as consistent as possible within the type of sample and the species of animal. For example, for cattle, non-limiting examples of sample sizes/methods include non-fatty meat: 0.0002 g to about 0.0010 g; hide: 0.0004 g to about 0.0010 g; hair roots: greater than five and less than twenty; buccal swabs: 15 to 20 seconds of rubbing with modest pressure in the area between outer lip and gum using one Cytosoft® cytology brush; bone: 0.0020 g to about 0.0040 g; blood: about 30 μl to about 70 μl.

Generally, the tissue sample is placed in a container that is labeled using a numbering system bearing a code corresponding to the animal, for example, to the animal's ear tag. Accordingly, the genotype of a particular animal is easily traceable at all times.

The tissue sample is then treated by the desired methods to retrieve the desired data, for example, such as fat content or genotype. Alternatively, the samples can be frozen for preservation and archived, for example, in the factory/slaughterhouse or a central storage location for future extraction/analysis as required.

In an advantageous embodiment of the invention, a sampling device and/or container is supplied to the farmer, a slaughterhouse, retailer or veterinarian. The sampling device advantageously takes a consistent and reproducible sample from individual animals while simultaneously avoiding any cross-contamination of tissue. Accordingly, the size and volume of sample tissues derived from individual animals would be consistent.

In the present invention, a sample of genomic DNA is obtained from a livestock. Generally, peripheral blood cells are used as the source of the DNA. A sufficient amount of cells are obtained to provide a sufficient amount of DNA for analysis. This amount will be known or readily determinable by those skilled in the art. The DNA is isolated from the blood cells by techniques known to those skilled in the art (see, e.g., U.S. Pat. Nos. 6,548,256 and 5,989,431, Hirota et al., Jinrui Idengaku Zasshi. September 1989;34(3):217-23 and John et al., Nucleic Acids Res. Jan. 25, 1991;19(2):408; the disclosures of which are incorporated by reference in their entireties).

In the method of the present invention, the source of the test nucleic acid is not critical. For example, the test nucleic acid can be obtained from cells within a body fluid of the livestock or from cells constituting a body tissue of the subject. The particular body fluid from which the cells are obtained is also not critical to the present invention. For example, the body fluid may be selected from the group consisting of blood, ascites, pleural fluid and spinal fluid. Furthermore, the particular body tissue from which cells are obtained is also not critical to the present invention. For example, the body tissue may be selected from the group consisting of skin, endometrial, uterine and cervical tissue. Both normal and tumor tissues can be used. Further, the source of the target material may include RNA or mitochondrial DNA.

The invention further comprises methods of screening livestock to determine those having predictably more uniform fat deposition based upon the presence or absence of certain polymorphisms in the ob gene. In an advantageous embodiment, the ob gene polymorphism is a C to T transition that results in an Arg25Cys in the leptin protein. One of ordinary skill in the art can apply the methods described herein for detecting polymorphisms of the ob gene to detect any other genotypes or polymorphisms correlating to a particular phenotype. Indications of high densities of SNPs in defined regions in the bovine subspecies Bos taurus and Bos indicus as well as chickens have been found (reviewed in Vignal et al., Genet. Sel. Evol. 34 (2002) 275-305, the disclosure of which is incorporated by reference in its entirety).

Any ob gene corresponding to the animal of interest can be used to identify the polymorphism(s) of interest in the ob gene. The ob gene that has been mapped to chromosome 6 in mice (Friedman & Leibel, 1992, Cell 69: 217-220), chromosome 7q31.3 in humans (Isse et al., 1995, J. Bio. Chem. 270: 27728-27733) chromosome 4 in cattle (Stone et al. 1996, Mamm. Genome 7: 399-400), and chromosome 18 in swine (Neuenschwander et al., 1996, Anim. Genet. 27: 275-278; Saskai et al., 1996, Mamm. Genome 7: 471-471). Sequences have been determined for the ob gene from mice (Zhang et al., 1994, Nature 372: 425-432), cattle (U.S. Pat. No. 6,297,027 to Spurlock), pigs (U.S. Pat. No. 6,277,592 to Bidwell and Spurlock; Neuenschwander et al., 1996, Anim. Genet. 27: 275-278), and humans (U.S. Pat. No. 6,309,857 to Friedman et al.) and there is significant conservation among the sequences of ob DNAs and leptin polypeptides from those species (Bidwell et al. 1997, Anim. Endocrinol. 8: 191-206; Ramsay et al. 1998, J. Anim. Sci. 76: 484-490).

In an advantageous embodiment, the ob sequence is a cattle ob sequence with the nucleotide sequence 5′ TCTGAAGACCTGGATGCGGGTGGTAACGGAGCACGTGGGTGTTCTCGGAGATCG ACGATGTGCCACGTGTGGTTTCTTCTGTTTTCAGGCCCCAGAAGCCCATCCCGGG AAGGAAAATGCGCTGTGGACCCCTGTATCGATTCCTGTGGCTTTGGCCCTATCTGT CTTACGTGGAGGCTGTGCCCATCTGCAAGGTCCAGGATGACACCAAAACCCTCAT CAAGACAATTGTCACCAGGATCAATGACATCTCACACACGGTAGGGAGGGACTG GGAGACGAGGTAGAACCGTGGCCATCCCGTGGGGGACCCCAGAGGCTGGCGGAG GAGGCTGTGCAGCCTTGCACAGGGCCCCAGTGGCCTGGACGCCCCCCTGGCATAA AGACAGCTCCTCTCCTCCTCCACTTCCCTTGCCTCCCGCCTTCTCACTCTCCTCCCT CCCAGACCGGAATCCTAGTGCCCAGGCCCAGAAGGAGTCACAGAGGTCCTGGGG TCCCCTTGGCAGGTGGCCAGAACCCCAGCAGCAGTCCCTCTGGGCCTCCATCTCA TTTCTAGAATGTTTTAGTCGTTAGGCATTCTTCCTGCCTGGTAACTG 3′ (SEQ ID NO:1), which contains the single nucleotide polymorphism at position 189. In another advantageous embodiment, the ob sequence is a cattle ob sequence with the nucleotide sequence 5′ TCTGAAGACCTGGATGCGGGTGGTAACGGAGCACGTGGGTGTTCTCGGAGATCG ACGATGTGCCACGTGTGGTTTCTTCTGTTTTCAGGCCCCAGAAGCCCATCCCGGG AAGGAAAATGCGCTGTGGACCCCTGTATCGATTCCTGTGGCTTTGGCCCTATCTGT CTTACGTGGAGGCTGTGCCCATCCGCAAGGTCCAGGATGACACCAAAACCCTCAT CAAGACAATTGTCACCAGGATCAATGACATCTCACACACGGTAGGGAGGGACTG GGAGACGAGGTAGAACCGTGGCCATCCCGTGGGGGACCCCAGAGGCTGGCGGAG GAGGCTGTGCAGCCTTGCACAGGGCCCCAGTGGCCTGGACGCCCCCCTGGCATAA AGACAGCTCCTCTCCTCCTCCACTTCCCTTGCCTCCCGCCTTCTCACTCTCCTCCCT CCCAGACCGGAATCCTAGTGCCCAGGCCCAGAAGGAGTCACAGAGGTCCTGGGG TCCCCTTGGCAGGTGGCCAGAACCCCAGCAGCAGTCCCTCTGGGCCTCCATCTCA TTTCTAGAATGTTTTAGTCGTTAGGCATTCTTCCTGCCTGGTAACTG 3′ (SEQ ID NO:2), which does not contain the single nucleotide polymorphism at position 189. In another embodiment, the bovine ob nucleotide sequence can be selected from any one of the sequences corresponding to GenBank Accession Nos. AB003143, AB070368, AB070369, AE003406, AF120500, AF536174, AJ132764, AJ236854, AJ512638, AJ512639, AJ571671, AJ580799, AJ580800, AJ580801, AR171261, AR171262, AR171263, AR171264, AR171265, AY044438, AY138588, NM_{—}000594, NM_{—}000600, NM_{—}000758, NM_{—}173926, NM_{—}173928, NM_{—}174140, NM_{—}174216, NM_{—}180996, U50365, U62385, U65793, U83512 and Y11369.

In an advantageous embodiment, the ob sequence is a cattle ob sequence with the amino acid sequence MRCGPLYRFLWLWPYLSYVEAVPIRKVQDDTKTLIKTIVTRINDISHTQSVSSKQRVT GLDFIPGLHPLLSLSKMDQTLAIYQQILTSLPSRNVVQISNDLENLRDLLHLLAASKSC PLPQVRALESLESLGVVLEASLYSTEVVALSRLQGSLQDMLRQLDLSPGC (SEQ ID NO:3). In another embodiment, the bovine ob amino acid sequence can be selected from any one of the sequences corresponding to Entrez Protein Accession Nos. AAE82807, AAK95823, AAN04050, AAN28921, BAA19750, BAB63371, CAA72197, CAB38018, CAB64255, CAD54745, CAE45337, CAE45338, CAE45339, NP_{—}000585, NP_{—}000591, NP_{—}000749, NP_{—}776351, NP_{—}776353, NP_{—}776565, NP_{—}776641, NP_{—}851339, P50595 and Q9BEG9, the disclosures of which are incorporated by reference in their entireties.

In an embodiment wherein the ob sequence is an ovine ob sequence, the ovine ob nucleotide sequence can be selected from any one of the sequences corresponding to GenBank Accession Nos. AF310264, AF118636 and U63719 and the ovine ob amino acid sequence can be selected from any one of the sequences corresponding to Entrez Protein Accession Nos. AAB51695, AAD17249, P79211, Q28602 and Q28603, the disclosures of which are incorporated by reference in their entireties.

In an embodiment wherein the ob sequence is an avian ob sequence, the avian ob nucleotide sequence can be selected from any one of the sequences corresponding to GenBank Accession Nos. NM_{—}012614, NT_{—}032977 and NW_{—}047717 and the avian ob amino acid can be selected from the sequence corresponding to Entrez Protein Accession No. NP_{—}036746, the disclosures of which are incorporated by reference in their entireties.

In an embodiment wherein the ob sequence is an swine ob sequence, the swine ob nucleotide sequence can be selected from any one of the sequences corresponding to GenBank Accession Nos. AF026976, AF036908, AF052691, AF092422, AF102856, AF167719, AF184172, AF184173, AF477386, AF477387, AH009271, AH011524, AJ223162, AJ223163, AY008846, AY079082, AY079083, U40812, U59894, U63540, U66254, U67739 and U72070 and the swine ob amino acid sequence can be selected from any one of the sequences corresponding to Entrez Protein Accession Nos. AAB06579, AAB40624, AAB61244, AAB62399, AAD23567, AAK95823, AAN04050, AAN28921, BAA19750, BAB63371, CAA72197, CAB38018, CAB64255, CAD54745, CAE45337, CAE45338, CAE45339, NP_{—}776351, NP_{—}776353, NP_{—}776565, NP_{—}776641, NP_{—}851339, P50595 and Q9BEG9, the disclosures of which are incorporated by reference in their entireties.

Also disclosed herein are oligonucleotides that can be used as primers to amplify specific nucleic acid sequences of the ob gene. The present invention also provides oligonucleotides that can be used as probes in the detection of amplified specific nucleic acid sequences of the ob gene. In certain embodiments, these probes and primers consist of oligonucleotide fragments. Such fragments should be of sufficient length to provide specific hybridization to an RNA or DNA tissue sample. The sequences typically will be about 8 to about 44 nucleotides, but may be longer. Longer sequences, e.g., from about 14 to about 50, are advantageous for certain embodiments.

Nucleic acid molecules having contiguous stretches of about 8, 9, 10, 11, 12, 13, 14, 15, 16, 17, 18, 19, 20, 21, 22, 23, or 24 nucleotides from a sequence selected from SEQ ID NO:4, SEQ ID NO:5, SEQ ID NO:6, and SEQ ID NO:7 are contemplated. Molecules that are complementary to the above mentioned sequences and that bind to these sequences under high stringency conditions also are contemplated. These probes will be useful in a variety of hybridization embodiments, such as Southern and Northern blotting. In some cases, it is contemplated that probes may be used that hybridize to multiple target sequences without compromising their ability to effectively detect the ob gene.

Various probes and primers can be designed around the disclosed nucleotide sequences. Primers may be of any length but, typically, are about 10 to about 24 bases in length. A probe or primer can be any stretch of at least 8, advantageously at least 10, more advantageously at least 12, 13, 14, or 15, such as at least 20, e.g., at least 23 or 25, for instance at least 27 or 30 nucleotides. As to PCR or hybridization primers or probes and optimal lengths therefor, reference is also made to Kajimura et al., GATA 7(4):71-79 (1990), the disclosure of which is incorporated by reference in its entirety. In certain embodiments, it is contemplated that multiple probes may be used for hybridization to a single sample. Designing and testing the probes and primers around the ob nucleotide sequences described above and from any one of the sequences corresponding to the accession numbers listed can be accomplished by one of ordinary skill in the art.

The use of a hybridization probe of between 10 and 30 nucleotides in length allows the formation of a duplex molecule that is both stable and selective. Molecules having complementary sequences over stretches greater than 12 bases in length are generally advantageous, in order to increase stability and selectivity of the hybrid, and thereby improve the quality and degree of particular hybrid molecules obtained. One will generally prefer to design nucleic acid molecules having stretches of 16 to 24 nucleotides, or even longer where desired. Such fragments may be readily prepared by, for example, directly synthesizing the fragment by chemical means or by introducing selected sequences into recombinant vectors for recombinant production.

In addition to leptin, the invention encompasses genetic testing of other genes and SNPs that correlate to a particular phenotype. One of ordinary skill in the art can easily apply the exemplified techniques described herein for leptin to other SNPs, genotypes and polymorphisms that correlate with a particular phenotype, physical characteristic or trait. For example, the design of an oligonucleotide primer to amplify a sequence (e.g., containing a genetic polymorphism of interest) of a given gene is routine experimentation for one of ordinary skill in the art. Such genes and/or SNPs include, but are not limited to, BGHR, calpain, calpastatin, CXCR2, DGAT1, FAA, TIMP2, IGF, IGF-2, POMC, neuropeptide Y, leptin receptor, thyroglobulin, UCP2 and UCP3.

In an embodiment wherein the gene of interest is bovine growth hormone receptor (“BGHR”), the BGHR nucleotide sequence can have the sequence corresponding to GenBank Accession No. X70041, or a fragment thereof, and the BGHR amino acid sequence can have the sequence corresponding to Entrez Protein Accession No. CAA49635, or a fragment thereof, the disclosures of which are incorporated by reference in their entireties.

In an embodiment wherein the gene of interest is bovine calpastatin, the bovine calpastatin nucleotide sequence can be selected from any one of the sequences corresponding to GenBank Accession Nos. AF071575, AF071576, AF071577, AF117813, AF159246, AF192536, AF281256, AF321530, AY008267, AY258325, BG224146, L14450, NM_{—}174003, U07859 and X67333, or a fragment thereof, and the bovine calpastatin amino acid sequence can be selected from any one of the sequences corresponding to Entrez Protein Accession Nos. A40432, AAA19643, AAC24460, AAD21054, AAD41621, AAD43339, AAF88058, AAG23869, AAG48625, AAP51021, B40432, CAA47748, NP_{—}776428, P20811, P51186, Q27970, Q27971, Q9TTH8 and S06638, or a fragment thereof, the disclosures of which are incorporated by reference in their entireties.

In an embodiment wherein the gene of interest is bovine chemokine receptor 2 (“CXCR2”), the bovine CXCR2 nucleotide sequence can have the sequence corresponding to GenBank Accession No. NM 002993, or a fragment thereof, and the bovine CXCR2 amino acid sequence can be selected from any one of the sequences corresponding to Entrez Protein Accession Nos. NP_{—}002984 and Q28003, or a fragment thereof, the disclosures of which are incorporated by reference in their entireties.

In an embodiment wherein the gene of interest, is bovine diacylglycerol O-acyltransferase 1 (“DGAT1”), the bovine DGAT1 nucleotide sequence can be selected from any one of the sequences corresponding to GenBank Accession Nos. AJ318490, AJ518948, AJ518949, AJ518950, AJ518951, AJ518952, AJ518953, AJ518954, AJ518955, AJ518956, AJ518957, AJ518958, AJ518959, AJ518960, AJ518961, AJ518962, AJ518963, AJ518964, AJ518965, AJ518966, AJ518967, AJ518968, AJ518969, AJ518970, AJ518971, AJ518972, AJ518973, AJ518974, AJ519351, AJ519352, AJ519353, AJ519354, AJ519355, AJ519356, AJ519357, AJ519358, AJ519359, AJ519360, AJ519361, AJ519362, AJ519363, AJ519780, AY065621 and NM_{—}174693, or a fragment thereof, and the bovine DGAT1 amino acid sequence can be selected from any one of the sequences corresponding to Entrez Protein Accession Nos. AAL49962, CAC86391, CAC86392, CAD58585, CAD58784, CAD58786, CAD58787, CAD58788, CAD58789, CAD58790, CAD58791, CAD58792, CAD58793, CAD58794, CAD58795, CAD58796, CAD58797, CAD58798, CAD58799, CAD58800, CAD58801, CAD58802, CAD58803, CAD58804, CAD58805, CAD58806, CAD58807, CAD58808, CAD58809, CAD58810, CAD58811 and NP_{—}777118, or a fragment thereof, the disclosures of which are incorporated by reference in their entireties.

In an embodiment wherein the gene of interest is bovine diacylglycerol O-acyltransferase 1 (“DGAT1”), the bovine DGAT1 nucleotide sequence can be selected from any one of the sequences corresponding to GenBank Accession Nos. AJ318490, AJ518948, AJ518949, AJ518950, AJ518951, AJ518952, AJ518953, AJ518954, AJ518955, AJ518956, AJ518957, AJ518958, AJ518959, AJ518960, AJ518961, AJ518962, AJ518963, AJ518964, AJ518965, AJ518966, AJ518967, AJ518968, AJ518969, AJ518970, AJ518971, AJ518972, AJ518973, AJ518974, AJ519351, AJ519352, AJ519353, AJ519354, AJ519355, AJ519356, AJ519357, AJ519358, AJ519359, AJ519360, AJ519361, AJ519362, AJ519363, AJ519780, AY065621 and NM_{—}174693, or a fragment thereof, and the bovine DGAT1 amino acid sequence can be selected from any one of the sequences corresponding to Entrez Protein Accession Nos. AAL49962, CAC86391, CAC86392, CAD58585, CAD58784, CAD58786, CAD58787, CAD58788, CAD58789, CAD58790, CAD58791, CAD58792, CAD58793, CAD58794, CAD58795, CAD58796, CAD58797, CAD58798, CAD58799, CAD58800, CAD58801, CAD58802, CAD58803, CAD58804, CAD58805, CAD58806, CAD58807, CAD58808, CAD58809, CAD58810, CAD58811 and NP_{—}777118, or a fragment thereof, the disclosures of which are incorporated by reference in their entireties.

In an embodiment wherein the gene of interest is bovine FAA (Fertility Associated Antigen) the bovine FAA nucleotide sequence can be selected from any one of the sequences corresponding to GenBank Accession Nos. BX294150, NC_{—}002677, NC_{—}003070, NC_{—}003197 and NC_{—}005027, or a fragment thereof, the disclosures of which are incorporated by reference in their entireties. In an embodiment wherein the gene of interest is bovine insulin-like growth factor (“IGF”), the bovine IGF nucleotide sequence can be selected from any one of the sequences corresponding to GenBank Accession Nos. NM_{—}000660, NM_{—}057387, NM_{—}001200, NM_{—}000876, NC_{—}003070, NM_{—}176608, NM_{—}001719, NM_{—}174603, NM_{—}174602, NM_{—}174555, NM_{—}173926, AY277406, AY277405, CD214364, CD214361, AJ320235, AJ320234, AF404761, U00668, U00667, U00666, U00665, U00664, U00663, U00659, AF226706, AF226707, AH009272, BM489874, BM489796, BM427639, Y18831, AF174576, S76122, AF017143, E01192, U33122, X53554, X53867, X15726 and X54980, or a fragment thereof, and the bovine IGF amino acid sequence can be selected from any one of the sequences corresponding to Entrez Protein Accession Nos. A60967, AAB52601, AAC03715, AAD14209, AAF66947, AA012197, AAP34700, AAP34701, CAA33746, CAA37861, CAA38724, CAC44343, CAC44344, IGBO1, IGBO2, NP_{—}000651, NP_{—}001191, NP_{—}001710, NP_{—}476735, NP_{—}776351, NP_{—}776980, NP_{—}777027, NP_{—}777028, NP_{—}788781, P07455, P07456, P10764, P13384, P20959, P24591, P82595, Q05688, Q05716, Q05717 and Q05718, or a fragment thereof, the disclosures of which are incorporated by reference in their entireties.

In an embodiment wherein the gene of interest is swine insulin-like growth factor (“IGF”), the swine IGF nucleotide sequence can be selected from any one of the sequences corresponding to GenBank Accession Nos. AB003362, AF085482, AF120326, AH006581, D84319, D84320, D84321, D84322, D84323, D84323, D84324, D84325, D84325, D84326, D84327, D84327, D84328, D84329, D84330, D84331, D84332, D84333, D84334, D84335, J05228, M31175, NM_{—}214003, NM_{—}214172, NM_{—}214256, U21117, U21118, U21119, U58370, U58650 and X64400, or a fragment thereof, and the swine IGF amino acid sequence can be selected from any one of the sequences corresponding to Entrez Protein Accession Nos. AAA31043, AAB02578, AAB02580, AAC48728, AAD33246, AAF23229, BAA19852, NP_{—}999168, NP_{—}999337, NP_{—}999421, P16545, P16611, P23695, P24853, P24854, Q28985, Q29000, Q29000_{—}1, Q29000_{—}2 and S12825, or a fragment thereof, the disclosures of which are incorporated by reference in their entireties.

In an embodiment wherein the gene of interest is bovine proopiomelanocortin (“POMC”), the bovine POMC nucleotide sequence can be selected from any one of the sequences corresponding to GenBank Accession Nos. AH005266, BG223864, J00014, J00015, J00016, J00018, J00019, J00020, J00021, J00291, J00610, J00611, J00612, J00759, M38606 and NM_{—}174151, or a fragment thereof, and the bovine POMC amino acid sequence can be selected from any one of the sequences corresponding to Entrez Protein Accession Nos. AAB24697, AAB59262, NP_{—}776576, P01190, P01191 and P21252, or a fragment thereof, the disclosures of which are incorporated by reference in their entireties.

In an embodiment wherein the gene of interest is bovine thyroglobulin, the bovine thyroglobulin nucleotide sequence can be selected from any one of the sequences corresponding to GenBank Accession Nos. AF036687, AY615525, BU917345, M16448, M21749, M35823, NM_{—}057605, NM_{—}079427, NM_{—}173883, U50312, X02155, X02815, X05380, X05381, X06071, X06072, X06073, X06074, X06075, X14324, X14325, Z46725, Z46726, Z46730 and Z92815, or a fragment thereof, and the bovine thyroglobulin amino acid sequence can be selected from any one of the sequences corresponding to Entrez Protein Accession Nos. 1101218A, 1109240A, 1405285A, A45403, A60967, AAA30777, AAA92322, AAB88311, AA032804, B32735, C32735, C45403, CAA26090, CAA26584, CAA28971, CAA29457, CAA32504, CAB07294, CAE82046, JN0064, NP_{—}476953, NP_{—}524151, NP_{—}776308, NP_{—}776979, NP_{—}776980, NP_{—}776981, NP_{—}776982, P01267, P13384, P20959, P24591, Q05716, Q05717, Q05718, S23009, UIBO and UIHU, or a fragment thereof, the disclosures of which are incorporated by reference in their entireties.

In an embodiment wherein the gene of interest is bovine uncoupling protein (“UCP”), the bovine UCP nucleotide sequence can be selected from any one of the sequences corresponding to GenBank Accession Nos. AF092048, AF127029, AF127030, AY147821 and NM_{—}174210, or a fragment thereof, and the bovine UCP amino acid sequence can be selected from any one of the sequences corresponding to Entrez Protein Accession Nos. AAC61762, AAD29672, AAD33339, AAO03556 and NP_{—}776635, or a fragment thereof, the disclosures of which are incorporated by reference in their entireties.

Methods for making a vector or recombinants or plasmid for amplification of the fragment either in vivo or in vitro can be any desired method, e.g., a method which is by or analogous to the methods disclosed in, or disclosed in documents cited in: U.S. Pat. Nos. 4,603,112; 4,769,330; 4,394,448; 4,722,848; 4,745,051; 4,769,331; 4,945,050; 5,494,807; 5,514,375; 5,744,140; 5,744,141; 5,756,103; 5,762,938; 5,766,599; 5,990,091; 5,174,993; 5,505,941; 5,338,683; 5,494,807; 5,591,639; 5,589,466; 5,677,178; 5,591,439; 5,552,143; 5,580,859; 6,130,066; 6,004,777; 6,130,066; 6,497,883; 6,464,984; 6,451,770; 6,391,314; 6,387,376; 6,376,473; 6,368,603; 6,348,196; 6,306,400; 6,228,846; 6,221,362; 6,217,883; 6,207,166; 6,207,165; 6,159,477; 6,153,199; 6,090,393; 6,074,649; 6,045,803; 6,033,670; 6,485,729; 6,103,526; 6,224,882; 6,312,682; 6,348,450 and 6,312,683; U.S. patent application Ser. No. 920,197, filed Oct. 16, 1986; WO 90/01543; W091/11525; WO 94/16716; WO 96/39491; WO 98/33510; EP 265785; EP 0 370 573; Andreansky et al., Proc. Natl. Acad. Sci. USA 1996;93:11313-11318; Ballay et al., EMBO J. 1993;4:3861-65; Felgner et al., J. Biol. Chem. 1994;269:2550-2561; Frolov et al., Proc. Natl. Acad. Sci. USA 1996;93:11371-11377; Graham, Tibtech 1990;8:85-87; Grunhaus et al., Sem. Virol. 1992; 3:237-52; Ju et al., Diabetologia 1998;41:736-739; Kitson et al., J. Virol. 1991;65:3068-3075; McClements et al., Proc. Natl. Acad. Sci. USA 1996;93:11414-11420; Moss, Proc. Natl. Acad. Sci. USA 1996;93:11341-11348; Paoletti, Proc. Natl. Acad. Sci. USA 1996; 93:11349-11353; Pennock et al., Mol. Cell. Biol. 1984;4:399-406; Richardson (Ed), Methods in Molecular Biology 1995;39, “Baculovirus Expression Protocols,” Humana Press Inc.; Smith et al. (1983) Mol. Cell. Biol. 1983;3:2156-2165; Robertson et al., Proc. Natl. Acad. Sci. USA 1996;93:11334-11340; Robinson et al., Sem. Immunol. 1997;9:271; and Roizman, Proc. Natl. Acad. Sci. USA 1996;93:11307-11312.

Accordingly, the nucleotide sequences of the invention may be used for their ability to selectively form duplex molecules with complementary stretches of genes or RNAs or to provide primers for amplification of DNA or RNA from tissues. Depending on the application envisioned, one will desire to employ varying conditions of hybridization to achieve varying degrees of selectivity of probe towards target sequence.

For applications requiring high selectivity, one will typically desire to employ relatively stringent conditions to form the hybrids, e.g., one will select relatively low salt and/or high temperature conditions, such as provided by about 0.02 M to about 0.10 M NaCl at temperatures of about 50° C. to about 70° C. Such high stringency conditions tolerate little, if any, mismatch between the probe and the template or target strand, and would be particularly suitable for isolating specific genes or detecting specific mRNA transcripts. It is generally appreciated that conditions can be rendered more stringent by the addition of increasing amounts of formamide.

It will be understood that this invention is not limited to the particular probes disclosed herein and particularly is intended to encompass at least nucleic acid sequences that are hybridizable to the disclosed sequences or are functional sequence analogs of these sequences.

One embodiment of the present invention is directed to a nucleic acid sequences (oligonucleotides) useful as primers and/or probes in the detection of an ob gene polymorphism in specimens. Also, the present invention is directed to a method of detecting the presence of ob gene polymorphism in a specimen wherein the oligonucleotides of the present invention may be used to amplify target nucleic acid sequences of an ob gene polymorphism that may be contained within a livestock specimen, and/or to detect the presence or absence of amplified target nucleic acid sequences of the ob gene polymorphism. Respective oligonucleotides may be used to amplify and/or detect ob gene and ob gene nucleic acid sequences. By using the oligonucleotides of the present invention and according to the methods of the present invention, as few as one to ten copies of the ob gene polymorphism may be detected in the presence of milligram quantities of extraneous DNA.

One embodiment of the present invention is directed to ob gene-specific oligonucleotides that can be used to amplify sequences of ob gene DNA, and to subsequently determine if amplification has occurred, from DNA extracted from a livestock specimen. A pair of ob gene-specific DNA oligonucleotide primers are used to hybridize to ob gene genomic DNA that may be present in DNA extracted from a livestock specimen, and to amplify the specific segment of genomic DNA between the two flanking primers using enzymatic synthesis and temperature cycling. Each pair of primers are designed to hybridize only to the ob gene DNA to which they have been synthesized to complement; one to each strand of the double-stranded DNA. Thus, the reaction is specific even in the presence of microgram quantities of heterologous DNA. For the purposes of this description, the primer derived from the sequence of the positive strand of DNA will be referred to as the “positive (+) primer”, and the primer derived from the sequence of the negative strand will be referred to as the “negative (−) primer”. Sequences that may be used include the primers AGGGATGCCTGGACACAAGA (sense, SEQ ID NO:4) and ATTGCCACCACCAGCAGCACCA (antisense, SEQ ID NO:5) and the probes CATCTGCTATGCGAATGCTTTG (SEQ ID NO:6) and GCTAATTATATTGTAAGACA (SEQ ID NO:7).

In one embodiment, the present invention relates to a composition for the detection of ob gene polymorphisms, consisting essentially of at least one purified and isolated oligonucleotide consisting of a nucleic acid sequence which complements and specifically hybridizes to an ob gene nucleic acid molecule, wherein said sequence is selected from the group consisting of SEQ ID NO:4, SEQ ID NO:5, SEQ ID NO:6, and SEQ ID NO:7, and a nucleotide sequence which differs from SEQ ID NO:4, SEQ ID NO:5, SEQ ID NO:6, or SEQ ID NO:7, by a one base change or substitution therein.

In another embodiment, the present invention relates to a method of detecting the presence of an ob gene polymorphism in a sample comprising (a) contacting the sample with the above-described nucleic acid probe, under conditions such that hybridization occurs, and (b) detecting the presence of the probe bound to the DNA segment. In an advantageous embodiment, the ob gene polymorphism is a C to T transition that results in an Arg25Cys in the leptin protein.

In another embodiment, the present invention relates to a method of detecting the presence of an ob gene polymorphism in a sample comprising (a) contacting the sample with the above-described nucleic acid probe, under conditions such that hybridization occurs, and (b) detecting the presence of the probe bound to the DNA segment. In an advantageous embodiment, the ob gene polymorphism is a C to T transition that results in an Arg25Cys in the leptin protein.

The actual hybridization reaction represents one of the most important and central steps in the whole process. The hybridization step involves placing the prepared DNA sample in contact with a specific sensor probe at set optimal conditions for hybridization to occur between the target DNA sequence and probe.

In their most basic form, hybridization assays function by discriminating oligonucleotide probe sensors against matched and mismatched targets. Currently, a variety of methods are available for detection and analysis of the hybridization events. Depending on the sensor group (fluorophore, enzyme, radioisotope, etc.) used to label the DNA probe, detection and analysis are carried out fluorimetrically, calorimetrically, or by autoradiography. By observing and measuring emitted radiation, such as fluorescent radiation or particle emission, information may be obtained about the hybridization events.

The secondary and tertiary structure of a single stranded target nucleic acid may be affected by binding “helper” oligonucleotides in addition to “probe” oligonucleotides causing a higher Tm to be exhibited between the probe and target nucleic acid.

Methods are provided for the analysis and determination of SNPs in a genetic target. In this embodiment, both wild type and mutant alleles are distinguished, if present in a sample, at a single capture site by detecting the presence of hybridized allele-specific probes labeled with fluorophores sensitive to excitation at various wave lengths.

In one embodiment, a target nucleic acid is first amplified, such as by PCR or SDA. The amplified dsDNA product is then denatured and hybridized with a probe. The hybridization complex formed is then subjected to destabilizing conditions to differentiate and determination of the ob SNP.

In another embodiment, the present invention relates to a method of detecting the presence of an ob gene polymorphism in a sample comprising a) contacting the sample with the above-described nucleic acid probe, under conditions such that hybridization occurs, b) enzymatically amplifying a specific region of the ob gene nucleic acid molecules, and c) detecting the presence of the probe bound to the DNA segment. In an advantageous embodiment, the ob gene polymorphism is a C to T transition that results in an Arg25Cys in the leptin protein.

In another embodiment, the present invention relates to a method of detecting the presence of an ob gene polymorphism in a sample comprising a) contacting the sample with the oligonucleotide primer pair of SEQ ID NO:4 and SEQ ID NO:5 that under suitable conditions permitting hybridization of the oligonucleotides to the nucleic acid molecules of the ob gene, b) enzymatically amplifying a specific region of the ob gene nucleic acid molecules using the oligonucleotide pair of SEQ ID NO:4 and SEQ ID NO:5 to form nucleic acid amplification products, c) contacting the amplified target sequences from step be, is present, with hybridization probes comprising the oligonucleotide pair of SEQ ID NO:6 and SEQ ID NO:7, labeled with a detectable moiety under suitable conditions permitting hybridization of the labeled oligonucleotide probe to amplified target sequences, and d) detecting the presence of amplified target sequences by detecting the detectable moiety of the labeled oligonucleotide probe hybridized to amplified target sequences. In an advantageous embodiment, prior to performing the above method, the sample is treated to release nucleic acid molecules from cells in the sample. In another advantageous embodiment, the presence of the amplified target sequences hybridized labeled oligonucleotide probe correlates to the presence of an ob gene polymorphism in the sample. In an advantageous embodiment, the ob gene polymorphism is a C to T transition that results in an Arg25Cys in the leptin protein.

Any one of the methods commercially available may accomplish amplification of DNA. For example, the polymerase chain reaction may be used to amplify the DNA. Once the primers have hybridized to opposite strands of the target DNA, the temperature is raised to permit replication of the specific segment of DNA across the region between the two primers by a thermostable DNA polymerase. Then the reaction is thermocycled so that at each cycle the amount of DNA representing the sequences between the two primers is doubled, and specific amplification of the ob gene DNA sequences, if present, results.

Further identification of the amplified DNA fragment, as being derived from ob gene DNA, may be accomplished by liquid hybridization. This method utilizes one or more oligonucleotides labeled with detectable moiety as probes to specifically hybridize to the amplified segment of ob gene DNA. Detection of the presence of sequence-specific amplified ob gene DNA may be accomplished by simultaneous detection of the complex comprising the labeled oligonucleotide hybridized to the sequence-specific amplified ob gene DNA (“amplified target sequences”) with respect to the DNA amplification. Detection of the presence of sequence-specific amplified ob gene DNA may also be accomplished using a gel retardation assay with subsequent detection of the complex comprising the labeled oligonucleotide hybridized to the sequence-specific amplified ob gene DNA.

In such a enzymatic amplification reaction hybridization system of ob gene allele detection, a specimen of blood, CSF, amniotic fluid, urine, body secretions, or other body fluid is subjected to a DNA extraction procedure. High molecular weight DNA may be purified from blood cells, tissue cells, or virus particles (collectively referred to herein as “cells”) contained in the livestock specimen using proteinase (proteinase K) extraction and ethanol precipitation. DNA may be extracted from a livestock specimen using other methods known in the art. Then, for example, the DNA extracted from the livestock specimen is enzymatically amplified in the polymerase chain reaction using ob gene-specific oligonucleotides (SEQ ID NO:4 and SEQ ID NO:5) as primer pairs. Following amplification, ob gene-specific oligonucleotides (SEQ ID NO:6 and SEQ ID NO:7) labeled with an appropriate detectable label are hybridized to the amplified target sequences, if present.

The contents of the hybridization reaction are then analyzed for detection of the sequence-specific amplified ob gene DNA, if present in the DNA extracted from the livestock specimen. Thus, the oligonucleotides of the present invention have commercial applications in diagnostic kits for the detection of ob gene DNA in livestock specimens.

The test samples suitable for nucleic acid probing methods of the present invention include, for example, cells or nucleic acid extracts of cells, or biological fluids. The sample used in the above-described methods will vary based on the assay format, the detection method and the nature of the tissues, cells or extracts to be assayed. Methods for preparing nucleic acid extracts of cells are well known in the art and can be readily adapted in order to obtain a sample that is compatible with the method utilized.

In a related embodiment of the present invention, the ob gene-specific oligonucleotides may be used to amplify and detect ob gene polymorphisms from DNA extracted from a livestock specimen. In this embodiment, the oligonucleotides used as primers may be labeled directly with detectable moiety, or synthesized to incorporate the label molecule. Depending on the label molecule used, the amplification products can then be detected, for example, after binding onto an affinity matrix, using isotopic or calorimetric detection. In an advantageous embodiment, the ob gene polymorphism is a C to T transition that results in an Arg25Cys in the leptin protein.

In an advantageous embodiment of this invention, cyclic polymerase-mediated reactions are performed. In certain embodiments of this invention, these processes are accomplished by changing the temperature of the solution containing the templates, primers, and polymerase. In such embodiments, the denaturation step is typically accomplished by shifting the temperature of the solution to a temperature sufficiently high to denature the template. In some embodiments, the hybridization step and the extension step are performed at different temperatures. In other embodiments, however, the hybridization and extension steps are performed concurrently, at a single temperature.

In some embodiments, the cyclic polymerase-mediated reaction is performed at a single temperature, and the different processes are accomplished by changing non-thermal properties of the reaction. For example, the denaturation step can be accomplished by incubating the template molecules with a basic solution or other denaturing solution.

In advantageous embodiments, the percentage of template molecules that are duplicated in the cycle steps is e.g. 90%, 70%, 50%, 30%, or less. Such cycles may be as short as 10, 8, 6, 5, 4.5, 4, 2, 1, 0.5 minutes or less. In certain embodiments, the reaction comprises 2, 5, 10, 15, 20, 30, 40, 50, or more cycles.

Typically, the reactions described herein are repeated until a detectable amount of product is generated. Often, such detectable amounts of product are between about 10 ng and about 100 ng, although larger quantities, e.g. 200 ng, 500 ng, 1 mg or more can also, of course, be detected. In terms of concentration, the amount of detectable product can be from about 0.01 pmol, 0.1 pmol, 1 pmol, 10 pmol, or more.

Any of a variety of polymerases can be used in the present invention. For thermocyclic reactions, the polymerases are thermostable polymerases such as Taq, KlenTaq, Stoffel Fragment, Deep Vent, Tth, Pfu, Vent, and UlTma, each of which are readily available from commercial sources. Similarly, guidance for the use of each of these enzymes can be readily found in any of a number of protocols found in guides, product literature, and other sources.

For non-thermocyclic reactions, and in certain thermocyclic reactions, the polymerase will often be one of many polymerases commonly used in the field, and commercially available, such as DNA pol 1, Klenow fragment, T7 DNA polymerase, and T4 DNA polymerase. Guidance for the use of such polymerases can readily be found in product literature and in general molecular biology guides.

Those of skill in the art are aware of the variety of nucleotides available for use in the present reaction. Typically, the nucleotides will consist at least in part of deoxynucleotide triphosphates (dNTPs), which are readily commercially available. Parameters for optimal use of dNTPs are also known to those of skill, and are described in the literature. In addition, a large number of nucleotide derivatives are known to those of skill and can be used in the present reaction. Such derivatives include fluorescently labeled nucleotides, allowing the detection of the product including such labeled nucleotides, as described below. Also included in this group are nucleotides that allow the sequencing of nucleic acids including such nucleotides, such as dideoxynucleotides and boronated nuclease-resistant nucleotides, as described below. Other nucleotide analogs include nucleotides with bromo-, iodo-, or other modifying groups, which groups affect numerous properties of resulting nucleic acids including their antigenicity, their replicatability, their melting temperatures, their binding properties, etc. In addition, certain nucleotides include reactive side groups, such as sulfhydryl groups, amino groups, N-hydroxysuccinimidyl groups, that allow the further modification of nucleic acids comprising them.

An oligonucleotide sequence used as a detection probe may be labeled with a detectable moiety. Various labeling moieties are known in the art. Said moiety may, for example, either be a radioactive compound, a detectable enzyme (e.g. horse radish peroxidase (HRP)) or any other moiety capable of generating a detectable signal such as a calorimetric, fluorescent, chemiluminescent or electrochemiluminescent signal. Advantageous analysis systems wherein said labels are used are electrochemiluminescence (ECL) based analysis or enzyme linked gel assay (ELGA) based analysis.

In one class of embodiments of this invention, a detectable label is incorporated into a nucleic acid during at least one cycle of the reaction. Spectroscopic, photochemical, biochemical, immunochemical, electrical, optical or chemical means can detect such labels. Useful labels in the present invention include fluorescent dyes (e.g., fluorescein isothiocyanate, Texas red, rhodamine, and the like), radiolabels (e.g., ³H, ¹²⁵I, ³⁵S, ¹⁴C, ³²P, etc.), enzymes (e.g. horseradish peroxidase, alkaline phosphatase etc.) colorimetric labels such as colloidal gold or colored glass or plastic (e.g. polystyrene, polypropylene, latex, etc.) beads. The label is coupled directly or indirectly to a component of the assay according to methods well known in the art. As indicated above, a wide variety of labels are used, with the choice of label depending on sensitivity required, ease of conjugation with the compound, stability requirements, available instrumentation, and disposal provisions. Non-radioactive labels are often attached by indirect means. Polymerases can also incorporate fluorescent nucleotides during synthesis of nucleic acids.

Reagents allowing the sequencing of reaction products can be utilized herein. For example, chain-terminating nucleotides will often be incorporated into a reaction product during one or more cycles of a reaction. Commercial kits containing the reagents most typically used for these methods of DNA sequencing are available and widely used. PCR exonuclease digestion methods for DNA sequencing can also be used.

Typically, the amplification sequence is serially diluted and then quantitatively amplified via the DNA Tag polymerase using a suitable PCR amplification technique. In PCR, annealing of the primers to the amplification sequence is generally carried out at about 37-50° C.; extension of the primer sequence by Taq polymerase in the presence of nucleoside triphosphates is carried out at about 70-75° C.; and the denaturing step to release the extended primer is carried out at about 90-95° C. In the two temperature PCR technique, the annealing and extension steps may both be carried at about 60-65° C., thus reducing the length of each amplification cycle and resulting in a shorter assay time.

Polymerase chain reactions (PCR) are generally carried out in about 25-50 μl samples containing 0.01 to 1.0 ng of template amplification sequence, 10 to 100 pmol of each generic primer, 1.5 units of Tag DNA polymerase (Promega Corp.), 0.2 mM DATP, 0.2 mM dCTP, 0.2 mM dGTP, 0.2 mM dTTP, 15 mM MgCl₂, 10 mM Tris-HCl (pH 9.0), 50 mM KCl, 1 μg/ml gelatin, and 10 μl/ml Triton X-100 (Saiki, 1988). Reactions are incubated at 94° C. for 1 minute, about 37 to 55° C. for 2 minutes (depending on the identity of the primers), and about 72° C. for about 3 minutes and repeated for about 5-40, cycles. A two temperature PCR technique differs from the above only in carrying out the annealing/extension steps at a single temperature, e.g., about 60-65° C. for about 5 minutes, rather than at two temperatures.

Another embodiment of the present invention is directed to ob gene-specific oligonucleotides that can be used to amplify sequences of ob gene DNA, and to subsequently determine if amplification has occurred, from DNA extracted from a livestock specimen. A pair of ob gene-specific DNA oligonucleotide primers are used to hybridize to ob gene genomic DNA that may be present in DNA extracted from a livestock specimen, and to amplify the specific segment of genomic DNA between the two flanking primers using enzymatic synthesis and temperature cycling. Each pair of primers are designed to hybridize only to the ob gene DNA to which they have been synthesized to complement; one to each strand of the double-stranded DNA. The region to which the primers have been synthesized to complement is conserved in ob gene. Thus, the reaction is specific even in the presence of microgram quantities of heterologous DNA.

A nucleic acid molecule of the present invention, e.g., a nucleic acid molecule having the nucleotide sequence of SEQ ID NO:4, SEQ ID NO:5, SEQ ID NO:6 or SEQ ID NO:7, or a complement thereof, can be isolated using standard molecular biology techniques and the sequence information provided herein. Using all or a portion of the nucleic acid sequence of SEQ ID NO:4, SEQ ID NO:5, SEQ ID NO:6 or SEQ ID NO:7, as a hybridization probe, nucleic acid sequences can be isolated using standard hybridization and cloning techniques. Furthermore, oligonucleotides can be prepared by standard synthetic techniques, e.g., using an automated DNA synthesizer.

In another embodiment, an isolated nucleic acid molecule of the invention comprises a nucleic acid molecule that is a complement of the nucleotide sequence shown in SEQ ID NO:4, SEQ ID NO:5, SEQ ID NO:6 or SEQ ID NO:7 or a portion of this nucleotide sequence. A nucleic acid molecule that is complementary to the nucleotide sequence shown in SEQ ID NO:4, SEQ ID NO:5, SEQ ID NO:6 or SEQ ID NO:7, is one that is sufficiently complementary to the nucleotide sequence shown in SEQ ID NO:4, SEQ ID NO:5, SEQ ID NO:6 or SEQ ID NO:7 that it can bind with few or no mismatches to the nucleotide sequence shown in SEQ ID NO:4, SEQ ID NO:5, SEQ ID NO:6 or SEQ ID NO:7, thereby forming a stable duplex.

A nucleic acid molecule of the invention may include only a fragment of the nucleic acid sequence of SEQ ID NO:4, SEQ ID NO:5, SEQ ID NO:6 or SEQ ID NO:7. Fragments provided herein are defined as sequences of at least 6 (contiguous) nucleic acids, a length sufficient to allow for specific hybridization of nucleic acids, and are at most some portion less than a full-length sequence. Fragments may be derived from any contiguous portion of a nucleic acid sequence of choice. Derivatives are nucleic acid sequences formed from the native compounds either directly or by modification or partial substitution. Analogs are nucleic acid sequences that have a structure similar to, but not identical to, the native compound but differ from it in respect to certain components or side chains. Analogs may be synthetic or from a different evolutionary origin and may have a similar or opposite metabolic activity compared to wild type.

Derivatives and analogs may be full length or other than full length, if the derivative or analog contains a modified nucleic acid or amino acid, as described below. Derivatives or analogs of the nucleic acids of the invention include, but are not limited to, molecules comprising regions that are substantially homologous to the nucleic acids of the invention, in various embodiments, by at least about 70%, 80%, 85%, 90%, 95%, 96%, 97%, 98%, or even 99% identity (with an advantageous identity of 80-99%) over a nucleic acid sequence of identical size or when compared to an aligned sequence in which the alignment is done by a computer homology program known in the art. Derivatives or analogs of the nucleic acids of the invention also include, but are not limited to, molecules comprising regions that are substantially homologous to the nucleic acids of the invention, in various embodiments, by at least about 70%, 80%, 85%, 90%, 95%, 96%, 97%, 98%, or even 99% identity (with an advantageous identity of 80-99%) under stringent, moderately stringent, or low stringent conditions.

For the purposes of the present invention, sequence identity or homology is determined by comparing the sequences when aligned so as to maximize overlap and identity while minimizing sequence gaps. In particular, sequence identity may be determined using any of a number of mathematical algorithms. A nonlimiting example of a mathematical algorithm used for comparison of two sequences is the algorithm of Karlin & Altschul, Proc. Natl. Acad. Sci. USA 1990;87: 2264-2268, modified as in Karlin & Altschul, Proc. Natl. Acad. Sci. USA 1993;90: 5873-5877.

Another example of a mathematical algorithm used for comparison of sequences is the algorithm of Myers & Miller, CABIOS 1988;4: 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson & Lipman, Proc. Natl. Acad. Sci. USA 1988;85: 2444-2448.

Advantageous for use according to the present invention is the WU-BLAST (Washington University BLAST) version 2.0 software. WU-BLAST version 2.0 executable programs for several UNIX platforms can be downloaded from ftp://blast.wustl. edu/blast/executables. This program is based on WU-BLAST version 1.4, which in turn is based on the public domain NCBI-BLAST version 1.4 (Altschul & Gish, 1996, Local alignment statistics, Doolittle ed., Methods in Enzymology 266: 460-480; Altschul et al., Journal of Molecular Biology 1 990;215: 403-410; Gish & States, 1 993;Nature Genetics 3: 266-272; Karlin & Altschul, 1993;Proc. Natl. Acad. Sci. USA 90: 5873-5877; all of which are incorporated by reference herein).

In all search programs in the suite the gapped alignment routines are integral to the database search itself. Gapping can be turned off if desired. The default penalty (Q) for a gap of length one is Q=9 for proteins and BLASTP, and Q=10 for BLASTN, but may be changed to any integer. The default per-residue penalty for extending a gap (R) is R=2 for proteins and BLASTP, and R=10 for BLASTN, but may be changed to any integer. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

Alternatively or additionally, the term “homology” or “identity”, for instance, with respect to a nucleotide or amino acid sequence, can indicate a quantitative measure of homology between two sequences. The percent sequence homology can be calculated as (N_{ref}−N_{dif})*100/N_{ref}, wherein N_{dif} is the total number of non-identical residues in the two sequences when aligned and wherein N_{ref} is the number of residues in one of the sequences. Hence, the DNA sequence AGTCAGTC will have a sequence identity of 75% with the sequence AATCAATC (N_{ref}=8; N_{dif}=2).

Alternatively or additionally, “homology” or “identity” with respect to sequences can refer to the number of positions with identical nucleotides or amino acids divided by the number of nucleotides or amino acids in the shorter of the two sequences wherein alignment of the two sequences can be determined in accordance with the Wilbur and Lipman algorithm (Wilbur & Lipman, Proc Natl Acad Sci USA 1983;80:726, incorporated herein by reference), for instance, using a window size of 20 nucleotides, a word length of 4 nucleotides, and a gap penalty of 4, and computer-assisted analysis and interpretation of the sequence data including alignment can be conveniently performed using commercially available programs (e.g., Intelligenetics™ Suite, Intelligenetics Inc. CA). When RNA sequences are said to be similar, or have a degree of sequence identity or homology with DNA sequences, thymidine (T) in the DNA sequence is considered equal to uracil (U) in the RNA sequence. Thus, RNA sequences are within the scope of the invention and can be derived from DNA sequences, by thymidine (T) in the DNA sequence being considered equal to uracil (U) in RNA sequences.

And, without undue experimentation, the skilled artisan can consult with many other programs or references for determining percent homology.

The nucleotide sequence of probes and primers typically comprises a substantially purified oligonucleotide. The oligonucleotide typically comprises a region of nucleotide sequence that hybridizes under stringent conditions to at least about 6, 9, 12, 16, 24, or more consecutive sense strand nucleotide sequence of SEQ ID NO:4, SEQ ID NO:5, SEQ ID NO:6 or SEQ ID NO:7, or an anti-sense strand nucleotide sequence of SEQ ID NO:4, SEQ ID NO:5, SEQ ID NO:6 or SEQ ID NO:7, or of a naturally occurring mutant of SEQ ID NO:4, SEQ ID NO:5, SEQ ID NO:6 or SEQ ID NO:7.

In various embodiments, the probe further comprises a label group attached thereto. Such probes can be used as a part of a diagnostic test kit for assessing the presence of homozygous mutant alleles of the ob gene (ob⁻/ob⁻ or TT animals), heterozygous mutant alleles of the ob gene (ob⁻/ob⁺ or CT animals) and wild-type alleles of the ob gene (ob⁺/ob⁺ or CC animals).

The invention further encompasses nucleic acid molecules that differ from the nucleotide sequences shown in SEQ ID NO:4, SEQ ID NO:5, SEQ ID NO:6 or SEQ ID NO:7, due to the degeneracy of the genetic code.

In addition to the nucleotide sequence shown in SEQ ID NO:4, SEQ ID NO:5, SEQ ID NO:6 or SEQ ID NO:7, it will be appreciated by those skilled in the art that DNA sequence polymorphisms in the ob gene DNA may exist within a population. Such natural allelic variations can typically result in about 1-5% variance in the nucleotide sequence of the gene. Any and all such nucleotide variations are intended to be within the scope of the invention.

Moreover, nucleic acid molecules that differ from the sequence of SEQ ID NO:4, SEQ ID NO:5, SEQ ID NO:6 or SEQ ID NO:7, are intended to be within the scope of the invention. Nucleic acid molecules corresponding to natural allelic variants and homologues of the DNAs of the invention can be isolated based on their homology to the nucleic acids disclosed herein using standard hybridization techniques under stringent hybridization conditions. Advantageously, such variations will differ from the sequence of SEQ ID NO:4, SEQ ID NO:5, SEQ ID NO:6 or SEQ ID NO:7, by only one nucleotide.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO:4, SEQ ID NO:5, SEQ ID NO:6 or SEQ ID NO:7.

Homologs (i.e., nucleic acids derived from other species) or other related sequences (e.g., paralogs) can be obtained under conditions of standard or stringent hybridization conditions with all or a portion of the particular sequence as a probe using methods well known in the art for nucleic acid hybridization and cloning.

In another embodiment, a nucleic acid sequence that is hybridizable to the nucleic acid molecule comprising the nucleotide sequence of SEQ ID NO:4, SEQ ID NO:5, SEQ ID NO:6 or SEQ ID NO:7, or fragments, analogs or derivatives thereof, under conditions of standard or stringent hybridization conditions is provided.

In addition to naturally-occurring allelic variants of the nucleotide sequence, the skilled artisan will further appreciate that changes can be introduced by mutation into the nucleotide sequence of SEQ ID NO:4, SEQ ID NO:5, SEQ ID NO:6 or SEQ ID NO:7.

In one embodiment, the isolated nucleic acid molecule comprises a nucleotide sequence at least about 75% homologous to the nucleotide sequence of SEQ ID NO:4, SEQ ID NO:5, SEQ ID NO:6 or SEQ ID NO:7. Advantageously, the nucleic acid is at least about 80% homologous to the nucleotide sequence of SEQ ID NO:4, SEQ ID NO:5, SEQ ID NO:6 or SEQ ID NO:7, more advantageously at least about 90%, 95%, 96%, 97%, 98%, and most advantageously at least about 99% homologous to the nucleotide sequence of SEQ ID NO:4, SEQ ID NO:5, SEQ ID NO:6 or SEQ ID NO:7.

As already indicated above, and will be presented in the experimental part of the description, both the sensitivity and reliability of polymorphism detection is greatly improved using the oligonucleotides according to the present invention when compared to known methods used in this art.

It is understood that oligonucleotides consisting of the sequences of the present invention may contain minor deletions, additions and/or substitutions of nucleic acid bases, to the extent that such alterations do not negatively affect the yield or product obtained to a significant degree.

Test kits for assessing the presence of homozygous mutant alleles of the ob gene (ob⁻/ob⁻ or TT animals), heterozygous mutant alleles of the ob gene (ob⁻/ob⁺ or CT animals) and wild-type alleles of the ob gene (ob⁺/ob⁺ or CC animals) are also part of the present invention. A test kit according to the invention may comprise a pair of oligonucleotides according to the invention and a probe comprising an oligonucleotide according to the invention. Such a test kit may additionally comprise suitable amplification reagents such as DNA and or RNA polymerases and mononucleotides. Test kits that can be used with the method according to the invention may comprise the oligonucleotides according to the invention for the amplification and subsequent assessment of for the presence of homozygous mutant alleles of the ob gene (ob⁻/ob⁻ or TT animals), heterozygous mutant alleles of the ob gene (ob⁻/ob⁺ or CT animals) and wild-type alleles of the ob gene (ob⁺/ob⁺ or CC animals). An advantageous embodiment for the test kit comprises the oligonucleotides: SEQ ID NO:4 and SEQ ID NO:5 as primer pairs for the amplification, and oligonucleotides SEQ ID NO:6 or SEQ ID NO:7, for use with SEQ ID NO:4 and SEQ ID NO:5, provided with a detectable label, as probes.

A diagnostic test kit for detection of ob gene according to the compositions and methods of the present invention may include, in separate packaging, a lysing buffer for lysing cells contained in the specimen; at least one oligonucleotide primer pair (SEQ ID NO:4 and SEQ ID NO:5); enzyme amplification reaction components such as dNTPs, reaction buffer, and/or amplifying enzyme; and at least one oligonucleotide probe labeled with a detectable moiety (SEQ ID NO:6 or SEQ ID NO:7), or various combinations thereof.

The present invention further provides nucleic acid detection kits, including arrays or microarrays of nucleic acid molecules that are based on one or more of the sequences provided in SEQ ID NO:4, SEQ ID NO:5, SEQ ID NO:6, and SEQ ID NO:7. As used herein “Arrays” or “Microarrays” refers to an array of distinct polynucleotides or oligonucleotides synthesized on a solid or flexible support, such as paper, nylon or other type of membrane, filter, chip, glass slide, or any other suitable solid support. In one embodiment, the microarray is prepared and used according to the methods and devices described in U.S. Pat. Nos. 5,446,603; 5,545,531; 5,807,522; 5,837,832; 5,874,219; 6,114,122; 6,238,910; 6,365,418; 6,410,229; 6,420,114; 6,432,696; 6,475,808 and 6,489,159 and PCT Publication No. WO 01/45843 A2, the disclosures of which are incorporated by reference in their entireties.

Although the above methods are described in terms of the use of a single probe and a single set of primers, the methods are not so limited. One or more additional probes and/or primers can be used, if desired. Additional enzymes, constructed probes and primers can be determined through routine experimentation.

The reagents suitable for applying the methods of the invention may be packaged into convenient kits. The kits provide the necessary materials, packaged into suitable containers. Advantageously, the containers are also supports useful in performing the assay. At a minimum, the kit contains a reagent that identifies a polymorphism in the livestock ob gene that is associated with an increased weight gain. Advantageously, the reagent is a probe and/or PCR set (a set of primers, DNA polymerase and 4 nucleoside triphosphates) that hybridize with the livestock ob gene or a fragment thereof.

Advantageously, both the probe (or PCR set) and a restriction enzyme that cleaves the livestock ob gene in at least one place are included in the kit. In a particularly advantageous embodiment of the invention, the probe comprises the human ob gene, the livestock ob gene, or a gene fragment that has been labeled with a detectable entity. Advantageously, the kit further comprises additional means, such as reagents, for detecting or measuring the detectable entity or providing a control. Other reagents used for hybridization, prehybridization, DNA extraction, etc. may also be included, if desired.

The methods and materials of the invention may also be used more generally to evaluate livestock DNA, genetically type individual livestock, and detect genetic differences in livestock. In particular, a sample of livestock genomic DNA may be evaluated by reference to one or more controls to determine if a polymorphism in the ob gene is present. Any method for determining genotype can be used for determining the ob genotype in the present invention. Such methods include, but are not limited to, amplimer sequencing, DNA sequencing, fluorescence spectroscopy, FRET-based hybridization analysis, high throughput screening, mass spectroscopy (e.g., MALDI-TOF MS), microsatellite analysis, nucleic acid hybridization, polymerase chain reaction (PCR), RFLP analysis and size chromatography (e.g., capillary or gel chromatography), all of which are well known to one of skill in the art. In particular, methods for determining nucleotide polymorphisms, particularly single nucleotide polymorphisms, are described in U.S. Pat. Nos. 6,514,700; 6,503,710; 6,468,742; 6,448,407; 6,410,231; 6,383,756; 6,358,679; 6,322,980; 6,316,230; and 6,287,766 and reviewed by Chen and Sullivan, Pharmacogenomics J 2003;3(2):77-96, the disclosures of which are incorporated by reference in their entireties.

Advantageously, FRET analysis is performed with respect to the livestock ob gene, and the results are compared with a control. The control is the result of a FRET analysis of the livestock ob gene of a different livestock where the polymorphism of the livestock ob gene is known. Similarly, the estrogen receptor genotype of a livestock may be determined by obtaining a sample of its genomic DNA, conducting FRET analysis of the ob gene in the DNA, and comparing the results with a control. A gain, the control is the result of FRET analysis of the ob gene of a different livestock. The results genetically type the livestock by specifying the polymorphism in its ob genes. Finally, genetic differences among livestock can be detected by obtaining samples of the genomic DNA from at least two livestock, identifying the presence or absence of a polymorphism in the ob gene, and comparing the results.

These assays are useful for identifying the genetic markers relating to weight gain, as discussed above, for identifying other polymorphisms in the ob gene that may be correlated with other characteristics, and for the general scientific analysis of livestock genotypes and phenotypes.

The genetic markers, methods, and kits of the invention are also useful in a breeding program to improve feed conversion efficiency in a breed, line, or population of livestock. Continuous selection and breeding of livestock that are at least heterozygous and advantageously homozygous for a polymorphism associated with increased feed conversion efficiency would lead to a breed, line, or population having higher numbers of offspring in each litter of the females of this breed or line. Thus, the markers can be used as selection tools.

It is to be understood that the application of the teachings of the present invention to a specific problem or environment will be within the capabilities of one having ordinary skill in the art in light of the teachings contained herein. The examples of the products and processes of the present invention appear in the following examples.

Further, the invention provides a method of using oligonucleotide primers (SEQ ID No.2 & SEQ ID No 3) based on this DNA sequence in a polymerase chain reaction (PCR) assay to distinguish livestock animals homozygous for mutant alleles of the ob gene (ob⁻/ob⁻ or TT animals), which alleles encode an altered leptin, from livestock animals heterozygous for mutant alleles of the ob gene (ob⁻/ob⁺ or CT animals) and livestock animals homozygous for wild-type alleles of the ob gene (ob⁺/ob⁺ or CC animals).

In another embodiment, the invention provides a method of using primers having SEQ ID No.2 & SEQ ID No 3 based on this DNA sequence in a polymerase chain reaction (PCR) assay to distinguish livestock animals homozygous for mutant alleles of the ob gene (ob⁻/ob⁻ or TT animals), which alleles encode an altered leptin, from livestock animals heterozygous for mutant alleles of the ob gene (ob⁻/ob⁺ or CT animals) and livestock animals homozygous for wild-type alleles of the ob gene (ob⁺/ob⁺ or CC animals), wherein detection of the PCR amplified fragment is by detection of a radioactively labeled nucleotide that is incorporated into the PCR amplified product.

In yet another embodiment, a non-radioactively labeled nucleotide is incorporated into the PCR amplified product and detection is by colorimetry, chemiluminescence, or measurement of fluorescence.

In another embodiment, the method of detection is based on the use of flourescently labeled nucleotides in Fluorescence Resonance Energy Transfer (FRET) based detection systems including Taqman, Molecular Beacon, etc., which are familiar to those conversant with prior art.

The oligonucleotides in the present invention can be produced by a conventional production process for general oligonucleotides, It can be produced, for example, by a chemical synthesis process or by a microbial process which makes use of a plasmid vector, a phage vector or the like (Tetrahedron Letters, 22, 1859-1862, 1981; Nucleic Acids Research, 14, 6227-6245, 1986). Further, it is suitable to use a nucleic acid synthesizer currently available on the market.

To label an oligonucleotide with the fluorescent dye, one of conventionally-known labeling methods can be used (Nature Biotechnology, 14, 303-308, 1996; Applied and Environmental Microbiology, 63, 1143-1147, 1997; Nucleic Acids Research, 24, 4532-4535, 1996). Reversed phase chromatography or the like used to provide a nucleic acid probe for use in the present invention can purify the synthesized oligonucleotide, which is labeled with the fluorescent dye.

The nucleic acid probe according to the present invention can be prepared as described above. An advantageous probe form is one labeled with a fluorescent dye at the 3′ or 5′end and containing G or C as the base at the labeled end. If the 5′end is labeled and the 3′end is not labeled, the OH group on the C atom at the 3′-position of the 3′end ribose or deoxyribose may be modified with a phosphate group or the like although no limitation is imposed in this respect.

Inclusion of the nucleic acid probe according to the present invention in a kit for analyzing or determining polymorphism and/or mutation of a target nucleic acid or gene, therefore, makes it possible to suitably use the kit as a kit for the analysis or determination of the polymorphism and/or mutation of the target nucleic acid or gene.

The probe according to the present invention may be immobilized on a surface of a solid (support layer), for example, on a surface of a slide glass. In this case, the probe may advantageously be immobilized on the end not labeled with the fluorescent dye. The probe of this form is now called a “DNA chips”. These DNA chips can be used for monitoring gene expressions, determining base sequences, analyzing mutations or analyzing polymorphisms such as single nucleotide polymorphism (SNP). They can also be used as devices (chips) for determining nucleic acids.

In one aspect, during the hybridization of the nucleic acid target with the probes, stringent conditions may be utilized, advantageously along with other stringency affecting conditions, to aid in the hybridization. Detection by differential disruption is particularly advantageous to reduce or eliminate slippage hybridization among probes and target, and to promote more effective hybridization. In yet another aspect, stringency conditions may be varied during the hybridization complex stability determination so as to more accurately or quickly determine whether a SNP is present in the target sequence.

Thus, the present invention provides for a method of determining a polymorphism comprising (a) obtaining a nucleic acid sample; (b) hybridizing the nucleic acid sample with a probe, and (c) disrupting the hybridization to determine the level of disruption energies required wherein the sensor probe has a different disruption energy if there is a mutation in the homology between the original nucleic acid sequence and sensor probe for hybridization. In one example, there is a lower disruption energy, e.g., melting temperature, for an allele that harbors the mutation site, and a higher required energy for an allele with no mutation since the homology is 100% and therefore requires more energy to cause the hybridized target to dissociate.

Optionally, in step (b) a second (“anchor”) probe used. Generally, the anchor probe is not specific to either t or c allele, but hybridizes regardless whether there is a c or t allele. The anchor probe does not affect the disruption energy required to disassociate the hybridization complex but, instead, contains a complementary label for using with the first (“sensor”) probe.

Hybridization stability may be influenced by numerous factors, including thermoregulation, chemical regulation, as well as electronic stringency control, either alone or in combination with the other listed factors. Through the use of stringency conditions, in either or both of the target hybridization step or the sensor oligonucleotide stringency step, rapid completion of the process may be achieved. This is desirable to achieve properly indexed hybridization of the target DNA to attain the maximum number of molecules at a test site with an accurate hybridization complex. By way of example, with the use of stringency, the initial hybridization step may be completed in ten minutes or less, more advantageously five minutes or less, and most advantageously two minutes or less. Overall, the analytical process may be completed in less than half an hour.

As to detection of the hybridization complex, it is advantageous that the complex is labeled. Typically, in the step of determining hybridization of probe to target, there is a detection of the amount of labeled hybridization complex at the test site or a portion thereof. Any mode or modality of detection consistent with the purpose and functionality of the invention may be utilized, such as optical imaging, electronic imaging, use of charge-coupled devices or other methods of quantification. Labeling may be of the target, capture, or sensor. Various labeling may be by fluorescent labeling, colormetric labeling or chemiluminescent labeling. In yet another implementation, detection may be via energy transfer between molecules in the hybridization complex. In yet another aspect, the detection may be via fluorescence perturbation analysis. In another aspect the detection may be via conductivity differences between concordant and discordant sites.

In yet another aspect, detection can be carried out using mass spectrometry. In such method, no fluorescent label is necessary. Rather detection is obtained by extremely high levels of mass resolution achieved by direct measurement, for example, by time of flight or by electron spray ionization (ESI). Where mass spectrometry is contemplated, sensor probes having a nucleic acid sequence of 50 bases or less are advantageous.

In one advantageous embodiment, the detection is carried out by MALDI-TOF MS (see, e.g., http://www.bmb.leeds.ac.uk/bmaf/msl.html). This system is particularly suitable for the purity assessment of synthetic peptides and oligonucleotides, the analysis of recombinant proteins, peptide fingerprinting, assessment of peptide labelling reactions (e.g. fluorescent tags), etc.

The tolerance of this technique to contaminating substances renders it an extremely valuable tool in biomolecule characterization. Low concentrations of general biological buffers, salt solutions, detergents, etc. may be tolerated, although they do make analysis more difficult. Often, if sufficiently concentrated, samples can be diluted to minimize interference from contaminating substances. For proteins, a mass error of <0.5% may be expected (e.g. 100 Da error for a 20 kDa protein). For peptides and oligonucleotides, a mass error of <0.1% may be expected (e.g. 1 Da error in 1000 Da).

Peptide and protein samples should normally be prepared at a concentration of 1-50 pmol/μl (μM). Oligonucleotide samples should be at a higher concentration (20-500 pmol/μl), as should samples containing contaminating substances. A 20 μl sample is sufficient for analysis in most cases. The sample is mixed with a matrix compound to promote desorption and ionization. For example, a protein matrix solution contains 3,5-dimethoxy-4-hydroxycinnamic acid (10 mg/ml in 30% acetonitrile/0.1% TFA, v/v) and a peptide matrix solution contains α-cyano-4-hydroxycinnamic acid (10 mg/ml in ethanol/acetonitrile, 1:1, v/v). The sample (2 μl) is mixed with an equal volume of the appropriate matrix solution and 1-2 μl are spotted onto a stainless steel target and allowed to air-dry, forming a co-crystalline sample/matrix complex. This sample disc is then inserted into the instrument.

Proteins and peptides are generally analyzed in positive mode. The approach taken favors the production of protonated (M+H)⁺ ions, although in appropriate buffer conditions sodium (M+Na)⁺, potassium (M+K)⁺ or ammonium (M+NH₄)⁺ adducts may also be formed. Oligonucleotides are analyzed in negative mode, detecting deprotonated (M−H)⁻ ions. MALDI/TOF-MS favors the production of singly-charged ions, generating a mass/charge spectrum which resembles a chromatogram, making interpretation relatively simple.

Samples are analyzed on a Micromass TofSpec instrument, using an N₂ laser at 337 nm to desorb the solute molecules from the sample disc. Following insertion of the probe containing the sample disc and evacuation of the system to <5×10⁻⁶ mbar, a voltage of 22500V (for peptides and oligonucleotides) is established in the source region (25000V for proteins). The microchannel plate detector in the analyzer is set to 1800V. Upon firing of the pulsed laser, molecules of the sample and matrix desorb and ionize at the high potential in the source region. The large molar excess of matrix molecules encourages the transfer of laser energy to the sample molecules, thus causing their volatilization. Coarse laser energy is set to 20% for peptide and oligonucleotide samples or 50% for protein samples, with fine adjustment being varied for each sample. Peptides and oligonucleotides are analysed in reflectron mode, with a mass error of <0.1%. Proteins are analyzed in linear mode, with a mass error of <0.5%. Once a good quality signal has been obtained by optimizing the parameters, 20-30 laser shots are accumulated to produce a mass/charge (m/z) spectrum. The spectrum is generally calibrated externally using an appropriate standard analyzed in similar conditions.

In one mode, the hybridization complex is labeled and the step of determining amount of hybridization includes detecting the amounts of labeled hybridization complex at the test sites. The detection device and method may include, but is not limited to, optical imaging, electronic imaging, imaging with a CCD camera, integrated optical imaging, and mass spectrometry. Further, the detection, either labeled or unlabeled, is quantified, which may include statistical analysis. The labeled portion of the complex may be the target, the stabilizer, the sensor or the hybridization complex in toto. Labeling may be by fluorescent labeling selected from the group of, but not limited to, Cy3, Cy5, Bodipy Texas Red, Bodipy Far Red, Lucifer Yellow, Bodipy 630/650-X, Bodipy R6G-X and 5-CR 6G. Labeling may further be accomplished by colormetric labeling, bioluminescent labeling and/or chemiluminescent labeling. Labeling further may include energy transfer between molecules in the hybridization complex by perturbation analysis, quenching, electron transport between donor and acceptor molecules, the latter of which may be facilitated by double stranded match hybridization complexes. Optionally, if the hybridization complex is unlabeled, detection may be accomplished by measurement of conductance differential between double stranded and non-double stranded DNA. Further, direct detection may be achieved by porous silicon-based optical interferometry or by mass spectrometry.

The label may be amplified, and may include for example branched or dendritic DNA. If the target DNA is purified, it may be unamplified or amplified. Further, if the purified target is amplified and the amplification is an exponential method, it may be, for example, PCR amplified DNA or strand displacement amplification (SDA) amplified DNA. Linear methods of DNA amplification such as rolling circle or transcriptional runoff may also be used.

By way of example, following incubation of the sensor probes, discrimination is achieved by subjecting the complex to destabilizing conditions, e.g., heating the complex to about 4° C. below melting temperature of the perfectly matched sensor/amplicon in a low salt buffer (e.g., 50 mM NaPO4). For FRET, imaging is then performed using two different lasers, one corresponding to the fluorophore on the wild-type sensor and one to the fluorophore on the mutant sensor. From these signal intensities, backgrounds are subtracted and specific activities are taken into account. A determination of wild type and mutant signals is achieved from which the allelic compositions of the amplicon products are determined.

In one embodiment, the method comprises (a) contacting the target nucleic acid of interest with at least one sensor oligonucleotide, wherein the sensor oligonucleotide comprises a sequence complementary to at least a portion of the target nucleic acid of interest, wherein the sensor oligonucleotide hybridizes to the target nucleic acid at a position suspected of containing the ob gene polymorphism and (b) subjecting the captured target nucleic acid and hybridized sensor probe oligonucleotide to destabilizing conditions, wherein the destabilizing conditions are sufficient to cause the sensor oligonucleotide to dissociate under differing conditions depending upon the presence of the cc, ct or tt polymorphisms in the ob gene.

In another embodiment, the method further comprises (c) detecting the hybridization of the sensor oligonucleotide to the target nucleic acid under the varying destabilizing conditions, whereby the presence of the specific sequence in the target nucleic acid is determined.

In yet another embodiment, the method further comprises a preparatory step of amplifying one or more target nucleic acid sequences from the nucleic acids of a sample, wherein the amplicons become the target nucleic acids.

In one embodiment, the amplification step produces single stranded amplicons, which are then utilized as the single stranded target nucleic acids. In another embodiment, the amplification step produces double stranded amplicons, further comprising a step of subjecting the amplicons to denaturing conditions to form single stranded target nucleic acids.

In an alternate embodiment, the amplification step is by an amplification method selected from the group consisting of polymerase chain reaction (PCR), strand displacement amplification (SDA), nucleic acid sequence-based amplification (NASBA), rolling circle amplification, T7 mediated amplification, T3 mediated amplification, and SP6 mediated amplification.

In one embodiment, the method comprising a step of subjecting the target nucleic acids of the sample to denaturing conditions to form single stranded target nucleic acids.

In another embodiment, the detection of the hybridization of the sensor oligonucleotide is by the detection of a labeling moiety on the sensor oligonucleotide selected from the group consisting of fluorescent moieties, bioluminescent moieties, chemiluminescent moieties, and colorigenic moieties. Advantageously, the labeling moiety is a fluorescent moiety selected from the group consisting of fluorescein derivatives, BODIPYL dyes, rhodamine derivatives, Lucifer Yellow derivatives, and cyanine (Cy) dyes.

In an alternate embodiment, the destabilizing conditions are created by methods selected from the group consisting of making temperature adjustments, making ionic strength adjustments, making adjustments in pH, and combinations thereof.

In one embodiment, the method comprises (a) contacting a single stranded target nucleic acid of interest with (i) a first sensor oligonucleotide, wherein the first sensor oligonucleotide comprises a sequence complementary to at least a portion of the target nucleic acid of interest; (ii) further contacting the target nucleic acid with at least a second sensor oligonucleotide, wherein the second sensor oligonucleotide comprises a sequence complementary to at least a portion of the target nucleic acid of interest; (b) subjecting the target nucleic acid and hybridized sensor oligonucleotides to destabilizing conditions, wherein the destabilizing conditions are sufficient to cause the first and/or second sensor oligonucleotide to dissociate under different destabilizing conditions; and (c) detecting the hybridization of the first and second sensor oligonucleotide to the target nucleic acid, whereby the presence of the polymorphism in the target nucleic acid is determined. Advantageously, the first and second sensor oligonucleotides are differently labeled with first and second labeling moieties.

In detecting a polymorphism by differential melting temperature, the region surrounding the SNP is amplified by PCR or other amplification method. In another embodiment, a detectable label is incorporated into the system, either by use of a labeled primer, a labeled nucleotide, a labeled ribonucleotide, a labeled, modified nucleotide or a labeled, modified ribonucleotide. Alternatively, a label may be incorporated after selective hybridization has occurred, i.e. after the temperature has been raised to a degree whereby at least one of the fragments dissociates from the oligonucleotide probe.

The cleavage products are hybridized to oligonucleotide probes designed to maximize the difference in hybridization signal obtained from the two different alleles. For optimal detection of single-base pair mismatches, an about a 1° C. to about 10° C. difference in melting temperature is advantageous. When the temperature is raised above the melting temperature of a fragment-oligonucleotide duplex corresponding to one of the alleles, that allele will disassociate. The remaining fragment-oligonucleotide duplexes can then be analyzed for the incorporated label that identified the polymorphism.

The present invention provides methods for identifying the presence of one or more SNP allele in a diploid DNA sample. The detection occurs when there is a loss of florescence emitted by the sensor probe. The sensor probe acquires energy from the anchor probe once conditions are adequate for hybridization between the target (genomic) DNA and the anchor and sensor probe. Once hybridization occurs, the anchor probe transfers its florescence energy to the sensor probe, which only will emit a specific wavelength after it has acquired the energy from the anchor probe. Detection of the SNP occurs as the temperature is raised at a predetermined rate, and a reading is acquired from the florescent light emitted. If there is a presence of the mutation (SNP) the sensor probe will dissociate sooner, or at a lower temperature, since the homology between the genomic DNA and the sensor probe will be less than that of genomic DNA that does not harbor the SNP. The melt occurs lower in the case of the DNA with the SNP since the stability is compromised slightly. This occurs, obviously, on both chromosomes at the same time, thus yielding either a reading of two identical melting temperatures, or a reading of two different melting temperatures, being the heterozygote. The individuals that harbor two copies of the SNP, dubbed “tt” melt at approximately 54° C., and the individuals containing only wild type DNA (no SNP present), dubbed “cc”, melt at approximately 63° C.

In one embodiment, the leptin (ob) mutation is genotyped as “tt” if the sample melts only at a low temperature (generally, at about 54° C.), as “ct” if the sample melts at both a high and a low temperature (generally, about 54° C. and about 63° C.), and “cc” if it melts at only the high temperature (generally, about 63° C.). The melting temperatures are generally within about 4° C., advantageously within about 2° C.

In one embodiment of the invention, the oligonucleotide probes used in the above assays can be immobilized on a solid support such as, without limitation, microchips, microbeads, glass slides or any other such matrix, all of which are within the scope of this invention.

Using an assay of this type, a fluorescent labeled probe anneals to the denatured single strand When the probe hybridizes to any specific target sequence produced as a result of the amplification reaction, the reactive molecule absorbs emission energy from labeled nucleotides or donates energy to the labeled nucleotides by means of FET or FRET, thus changing the signal from the fluorescent nucleotides. Advantageously, the receptor probe takes the energy emitted from the donor probe and emits energy at a different wavelength, which is then measured. This new wavelength emission may be detected and this indicates binding of the probe. Alternatively, the reactive molecule is able to absorb fluorescence from the labeled nucleotides and so the fluorescence from these is reduced. This reduction may be detected and this indicates binding of the probe.

Most advantageously, the reactive molecule is an acceptor molecule which it emits fluorescence at a characteristic wavelength. In this case, increase in fluorescence from the acceptor molecule, which is of a different wavelength to that of the labeled nucleotide, will also indicate binding of the probe.

The presence of the labeled amplification product can be detected by monitoring fluorescence from the acceptor molecule on the probe, which specifically binds only the target sequence. In this case, signal from the amplification product can be distinguished from background signal of the fluorescent label and also from any non-specific amplification product.

An assay of this nature can be carried out using inexpensive reagents. Single labeled probes are more economical to those that include both acceptor and donor molecules.

As used herein, the expression “set of nucleotides” refers to a group of nucleotides that are sufficient to form nucleic acids such as DNA and RNA. Thus these comprise adenosine, cytosine, guanine and thymine or uracil. One or more of these is fluorescently labeled.

Amplification is suitably effected using known amplification reactions such as the polymerase chain reaction (PCR) or the ligase chain reaction (LCR), strand displacement assay (SDA) or NASBA, but advantageously PCR.

In some embodiments, the fluorescence of both the nucleotide and the acceptor molecule are monitored and the relationship between the emissions calculated.

Suitable reactive molecules (such as acceptor molecules) are rhodamine dyes or other dyes such as Cy5. These may be attached to the probe in a conventional manner. The position of the reactive molecule along the probe is immaterial although it general, they will be positioned at an end region of the probe.

In order for FET, such as FRET, to occur between the reactive molecule and fluorescent emission of the nucleotides, the fluorescent emission of the element (reactive molecule or labeled nucleotide) which acts as the donor must be of a shorter wavelength than the element acceptor. Suitable combinations are SYBRGold and rhodamine; SYBRGreen I and rhodamine; SYBRGold and Cy5; SYBRGreen I and Cy5; and fluorescein and ethidium bromide.

Advantageously, the molecules used as donor and/or acceptor produce sharp peaks, and there is little or no overlap in the wavelengths of the emission. Under these circumstances, it may not be necessary to resolve the “strand specific peak” from the signal produced by amplification product. A simple measurement of the strand specific signal alone (i.e. that provided by the reactive molecule) will provide information regarding the extent of the FET or FRET caused by the target reaction. The ethidium bromide/fluorescein combination may fulfill this requirement. In that case, the strand specific reaction will be quantifiable by the reduction in fluorescence at 640 nm, suitably expressed as 1/Fluorescence.

However, where there is a spectral overlap in the fluorescent signals from the donor and acceptor molecules, this can be accounted for in the results, for example by determining empirically the relationship between the spectra and using this relationship to normalize the signals from the two signals.

In one method of the invention, the sample may be subjected to conditions under which the probe hybridizes to the samples during or after the amplification reaction has been completed. The process allows the detection to be effected in a homogenous manner, in that the amplification and monitoring can be carried out in a single container with all reagents added initially. No subsequent reagent addition steps are required. Neither is there any need to effect the method in the presence of solid supports (although this is an option as discussed further hereinafter).

For example, where the probe is present throughout the amplification reaction, the fluorescent signal may allow the progress of the amplification reaction to be monitored. This may provide a means for quantitating the amount of target sequence present in the sample.

During each cycle of the amplification reaction, amplicon strands containing the target sequence bind to probe and thereby generate an acceptor signal. As the amount of amplicon in the sample increases, so the acceptor signal will increase. By plotting the rate of increase over cycles, the start point of the increase can be determined.

The probe may comprise a nucleic acid molecule such as DNA or RNA, which will hybridize to the target nucleic acid sequence when the latter is in single stranded form. In this instance, step (b) will involve the use of conditions which render the target nucleic acid single stranded. Alternatively, the probe may comprise a molecule such as a peptide nucleic acid that specifically binds the target sequence in double stranded form.

In particular, the amplification reaction used will involve a step of subjecting the sample to conditions under which any of the target nucleic acid sequence present in the sample becomes single stranded, such as PCR or LCR.

It is possible then for the probe to hybridize during the course of the amplification reaction provided appropriate hybridization conditions are encountered.

In an advantageous embodiment, the probe may be designed such that these conditions are met during each cycle of the amplification reaction. Thus at some point during each cycle of the amplification reaction, the probe will hybridize to the target sequence, and generate a signal as a result of the FET or FRET. As the amplification proceeds, the probe will be separated or melted from the target sequence and so the signal generated by the reactive molecule will either reduce or increase depending upon whether it comprises the donor or acceptor molecule. For instance, where it is an acceptor, in each cycle of the amplification, a fluorescence peak from the reactive molecule is generated. The intensity of the peak will increase as the amplification proceeds because more target sequence becomes available for binding to the probe.

By monitoring the fluorescence of the reactive molecule from the sample during each cycle, the progress of the amplification reaction can be monitored in various ways. For example, the data provided by melting peaks could be analyzed, for example by calculating the area under the melting peaks and this data plotted against the number of cycles.

The probe may either be free in solution or immobilized on a solid support, for example to the surface of a bead such as a magnetic bead, useful in separating products, or the surface of a detector device, such as the waveguide of a surface plasma resonance detector. The selection will depend upon the nature of the particular assay being looked at and the particular detection means being employed.

An increase in fluorescence of the acceptor molecule in the course of or at the end of the amplification reaction is indicative of an increase in the amount of the target sequence present, suggestive of the fact that the amplification reaction has proceeded and therefore the target sequence was in fact present in the sample.

Thus, one embodiment of the invention comprises a method for detecting nucleic acid amplification comprising: performing nucleic acid amplification on a target polynucleotide in the presence of (a) a nucleic acid polymerase (b) at least one primer capable of hybridizing to the target polynucleotide, (c) a set of nucleotides, at least one of which is fluorescently labeled and (d) an oligonucleotide probe which is capable of binding to the target polynucleotide sequence and which contains a reactive molecule which is capable of absorbing fluorescence from or donating fluorescence to the labeled nucleotide; and monitoring changes in fluorescence during the amplification reaction. Suitably, the reactive molecule is an acceptor molecule that can absorb energy from the labeled nucleotide.

The amplification is suitably carried out using a pair of primers which are designed such that only the target nucleotide sequence within a DNA strand is amplified as is well understood in the art. The nucleic acid polymerase is suitably a thermostable polymerase such as Taq polymerase.

Suitable conditions under which the amplification reaction can be carried out are well known in the art. The optimum conditions may be variable in each case depending upon the particular amplicon involved, the nature of the primers used and the enzymes employed. The optimum conditions may be determined in each case by the skilled person. Typical denaturation temperatures are of the order of 95° C., typical annealing temperatures are of the order of 55° C. and extension temperatures are of the order of 72° C. Alternatively or additionally, the method of the invention can be used in hybridization assays for determining characteristics of a sequence. Thus in a further aspect, the invention provides a method for determining a characteristic of a sequence, the method comprising (a) amplifying the sequence using a set of nucleotides, at least one of which is fluorescently labeled, (b) contacting amplification product with a probe under conditions in which the probe will hybridize to the target sequence, the probe comprising a reactive molecule which is able to absorb fluorescence from or donate fluorescent energy to the fluorescent labeled nucleotide and (c) monitoring fluorescence of the sample and determining a particular reaction condition, characteristic of the sequence, at which fluorescence changes as a result of the hybridization of the probe to the sample or destabilization of the duplex formed between the probe and the target nucleic acid sequence.

Suitable reaction conditions include temperature, electrochemical, or the response to the presence of particular enzymes or chemicals. By monitoring changes in fluorescence as these properties are varied, information characteristic of the precise nature of the sequence can be achieved. For example, in the case of temperature, the temperature at which the probe separates from the sequences in the sample as a result of heating can be determined.

Another way to produce a FRET signal that discriminates between the two variant alleles is to incorporate a nucleotide with a dye that interacts with the dye on-the primer. The key to achieving differential FRET is that the dye modified nucleotide must first occur (after the 3′ end of the primer) beyond the polymorphic site so that, after cleavage, the nucleotide dye of one allele (cleaved) will no longer be in within the requisite resonance producing distance of the primer dye while, in the other (uncleaved) allele, the proper distance will be maintained and FRET will occur.

In the present invention, the above-described nucleic acid probe is added to a measurement system and is caused to hybridize to a target nucleic acid. This hybridization can be by conventionally known methods. As conditions for hybridization, the salt concentration may range from 0 to 2 molar concentration, advantageously from 0.1 to 1.0 molar concentration, and the pH may range from 6 to 8, advantageously from 6.5 to 7.5.

The reaction temperature may advantageously be in a range of the Tm value of the hybrid complex, which is to be formed by hybridization of the nucleic acid probe to the specific site of the target nucleic acid, ±10° C. This temperature range can prevent non-specific hybridization. Reaction temperature lowers than Tm−10° C. allows non-specific hybridization, while a reaction temperature higher than Tm+10° C. allows no hybridization. Incidentally, a Tm value can be determined in a similar manner as in an experiment that is needed to design the nucleic acid probe for use in the present invention. Described specifically, an oligonucleotide which is to be hybridized with the nucleic acid probe and has a complementary base sequence to the nucleic acid probe is chemically synthesized by the above-described nucleic acid synthesizer or the like, and the Tm value of a hybrid complex between the oligonucleotide and the nucleic acid probe is then measured by a conventional method.

The reaction time may range from 1 second to 180 minutes, advantageously from 5 seconds to 90 minutes. If the reaction time is shorter than 1 second, a substantial portion of the nucleic acid probe according to the present invention remains unreacted in the hybridization. On the other hand, no particular advantage can be brought about even if the reaction time is set excessively long. The reaction time varies considerably depending on the kind of the nucleic acid, namely, the length or base sequence of the nucleic acid.

In the present invention, the nucleic acid probe is hybridized to the target nucleic acid as described above. The intensity of fluorescence emitted from the fluorescent dye is measured both before and after the hybridization, and a decrease in fluorescence intensity after the hybridization is then calculated. As the decrease is proportional to the concentration of the target nucleic acid, the concentration of the target nucleic acid can be determined.

In certain embodiments of the present invention, the detection of polymorphic sites in a target polynucleotide may be facilitated through the use of nucleic acid amplification methods. Such methods may be used to specifically increase the concentration of the target polynucleotide (i.e., sequences that span the polymorphic site, or include that site and sequences located either distal or proximal to it). Such amplified molecules can be readily detected by gel electrophoresis, or other means.

The most advantageous method of achieving such amplification employs PCR (see e.g., U.S. Pat. Nos. 4,965,188; 5,066,584; 5,338,671; 5,348,853; 5,364,790; 5,374,553; 5,403,707; 5,405,774; 5,418,149; 5,451,512; 5,470,724; 5,487,993; 5,523,225; 5,527,510; 5,567,583; 5,567,809; 5,587,287; 5,597,910; 5,602,011; 5,622,820; 5,658,764; 5,674,679; 5,674,738; 5,681,741; 5,702,901; 5,710,381; 5,733,751; 5,741,640; 5,741,676; 5,753,467; 5,756,285; 5,776,686; 5,811,295; 5,817,797; 5,827,657; 5,869,249; 5,935,522; 6,001,645; 6,015,534; 6,015,666; 6,033,854; 6,043,028; 6,077,664; 6,090,553; 6,168,918; 6,174,668; 6,174,670; 6,200,747; 6,225,093; 6,232,079; 6,261,431; 6,287,769; 6,306,593; 6,440,668; 6,468,743; 6,485,909; 6,511,805; 6,544,782; 6,566,067; 6,569,627; 6,613,560; 6,613,560 and 6,632,645; the disclosures of which are incorporated by reference in their entireties), using primer pairs that are capable of hybridizing to the proximal sequences that define or flank a polymorphic site in its double-stranded form.

In some embodiments of the present invention, the amplification method is itself a method for determining the identity of a polymorphic site, as for example, in allele-specific PCR. In allele-specific PCR, primer pairs are chosen such that amplification is dependent upon the input template nucleic acid containing the polymorphism of interest. In such embodiments, primer pairs are chosen such that at least one primer is an allele-specific oligonucleotide primer. In some sub-embodiments of the present invention, allele-specific primers are chosen so that amplification creates a restriction site, facilitating identification of a polymorphic site. In other embodiments of the present invention, amplification of the target polynucleotide is by multiplex PCR. Through the use of multiplex PCR, a multiplicity of regions of a target polynucleotide may be amplified simultaneously. This is particularly advantageous in those embodiments wherein greater than a single polymorphism is detected.

In lieu of PCR, alternative methods, such as the “Ligase Chain Reaction” (“LCR”) may be used (Barany, F., Proc. Natl. Acad. Sci. (U.S.A.) 88:189-193 (1991)). The “Oligonucleotide Ligation Assay” (“OLA”) (Landegren, U. et al., Science 241:1077-1080 (1988)) shares certain similarities with LCR and is also a suitable method for analysis of polymorphisms. Nickerson, D. A. et al. have described a nucleic acid detection assay that combines attributes of PCR and OLA (Nickerson, D. A. et al., Proc. Natl. Acad. Sci. (U.S.A.) 87:8923-8927 (1990)). Other known nucleic acid amplification procedures, such as transcription-based amplification systems (Malek, L. T. et al., U.S. Pat. No. 5,130,238; Davey, C. et al., European Patent Application 329,822; Schuster et al., U.S. Pat. No. 5,169,766; Miller, H. I. et al., PCT Application WO89/06700; Kwoh, D. et al., Proc. Natl. Acad. Sci. (U.S.A.) 86:1173 (1989); Gingeras, T. R. et al., PCT Application WO88/10315)), or isothermal amplification methods (Walker, G. T. et al., Proc. Natl. Acad. Sci. (U.S.A.) 89:392-396 (1992)) may also be used.

An advantageous gene, particularly its alleles, is the ob gene. Other genetic sequences include, but are not limited to, microsatellite loci for use in bovine parentage verification, including those designated ISAG markers, URB markers as developed by H. Lewin at the Bovine Blood Typing Lab, Saskatchewan Research Council, Saskatoon, Saskatchewan, Canada, and other species specific and genotype specific nucleotide sequences.

The invention also provides for the sequencing of the genetic sequences described above. Advantageously, the nucleic acid sequencing is by automated methods (reviewed by Meldrum, Genome Res. September 2000;10(9):1288-303, the disclosure of which is incorporated by reference in its entirety). Methods for sequencing nucleic acids include, but are not limited to, automated fluorescent DNA sequencing (see, e.g., Watts & MacBeath, Methods Mol Biol. 2001;167:153-70 and MacBeath et al., Methods Mol Biol. 2001;167:119-52), capillary electrophoresis (see, e.g., Bosserhoff et al., Comb Chem High Throughput Screen. December 2000; 3(6):455-66), DNA sequencing chips (see, e.g., Jain, Pharmacogenomics. August 2000; 1(3):289-307), mass spectrometry (see, e.g., Yates, Trends Genet. January 2000;16(1):5-8), pyrosequencing (see, e.g., Ronaghi, Genome Res. January 2001;11(1):3-11), and ultrathin-layer gel electrophoresis (see, e.g., Guttman & Ronai, Electrophoresis. December 2000;21(18):3952-64), the disclosures of which are hereby incorporated by reference in their entireties. The sequencing can also be done by any commercial company. Examples of such companies include, but are not limited to, the University of Georgia Molecular Genetics Instrumentation Facility (Athens, Georgia) or SeqWright DNA Technologies Services (Houston, Tex.).

Advantageously, the amino acid sequencing is by automated methods. Methods for sequencing amino acids include, but are not limited to, alkylated-thiohydantoin method (see, e.g., Dupont et al., EXS. 2000;88:119-31), chemical protein sequencing (see, e.g., Stolowitz, Curr Opin Biotechnol. February 1993;4(1):9-13), Edman degradation (see, e.g., Prabhakaran et al., J Pept Res. July 2000;56(1):12-23), and mass spectrometry (see, e.g., McDonald et al., Dis Markers. 2002;18(2):99-105), the disclosures of which are incorporated by reference in their entireties. Alternatively, amino acid sequences can be deduced from nucleic acid sequences. Such methods are well known in the art, e.g., EditSeq from DNASTAR, Inc.

The results of the analysis provide the genotype data that is associated with the individual animal from which the sample was taken. The genotype data is then kept in an accessible database, and may or may not be associated with other data from that particular individual or from other animals.

The data obtained from genotyping individual animals is stored in a database which can be integrated or associated with and/or cross-matched to other databases. The database along with the associated data allows information about the individual animal to be known through every stage of the animal's life, i.e., from conception to consumption of the animal product.

The accumulated data, the combination of the genetic data with other types of data of the animal provides access to information about parentage, identification of herd, health information including vaccinations, exposure to diseases, feed lot location, diet and ownership changes. Information such as dates and results of diagnostic or routine tests are easily stored and attainable. Such information would be especially valuable to companies specializing in artificial insemination of animals, particularly those who seek superior breeding lines.

Each animal is provided with a unique identifier. The animal can be tagged, as in traditional tracing programs or have implant computer chips providing stored and readable data or provided with any other identification method which associates the animal with its unique identifier.

The database containing the genotype results for each animal or the data for each animal can be associated or linked to other databases containing data, for example, which may be helpful in selecting traits for grouping or sub-grouping of an animal. For example, and not for limitation, data pertaining to animals grouped for propensity to lay fat can linked with data pertaining to animals having particular hormone levels, and optionally can be further linked with data pertaining to animals having food from certain food sources. The ability to refine a group of animals is limited only by the traits sought and the databases containing information related to those traits.

Databases with which the genotyping data can be associated include specific or general scientific data. Specific data includes, but is not limited to, breeding lines, sires, dames, and the like, other animals' genotypes, including whether or not other specific animals possess specific genes, location of animals which share similar or identical genetic characteristics, and the like. General data includes scientific data such as which genes encode for specific quality characteristics, breed association data, feed data, breeding trends, and the like.

A method of the present invention includes providing the animal owner or customer with sample collection equipment, such as swabs and vials. The vials are packaged in a container which is encoded with identifying indicia. Advantageously, the packaging is encoded with a bar code label. The vials are encoded with the same identifying indicia, advantageously with a matching bar code label. Optionally, the packaging contains means for sending the vials to a laboratory for analysis. The optional packaging is also encoded with identifying indicia, advantageously with a bar code label.

The method optionally includes a system wherein a database account is established upon ordering the sampling equipment. The database account identifier corresponds to the identifying indicia of the vials and the packaging. Upon shipment of the sampling equipment in fulfillment of the order, the identifying indicia are recorded in a database. Advantageously, the identifier is a bar code label which is scanned when the vials are sent. When the vials are returned to the testing facility, the identifier is again recorded and matched to the information previously recorded in the database upon shipment of the vial to the customer. Once the genotyping is completed, the information is recorded in the database and coded with the unique identifier. Test results are also provided to the customer or animal owner.

The data stored in the genotype database can be integrated with or compared to other data or databases for the purpose of identifying animals based on genetic propensities. Other data or databases include, but are not limited to, those containing information related to DNA testing, thyroglobulin testing, leptin, MMI (meta morphix Inc.), Bovine spongiform encephalopathy (BSE) diagnosis, brucellosis vaccination, FMD^{(Foot and Mouth Disease)} vaccination, BVD (bovine viral diarrhea) vaccination, SUREBRED pre-conditioning program, estrus and pregnancy results, tuberculosis, hormone levels, food safety/contamination, somatic cell counts, mastitis occurrence, diagnostic test results, milk protein levels, milk fat, vaccine status, health records, mineral levels, trace mineral levels, herd performance, and the like.

The present invention comprises methods for cattle wherein bulls are tested for any of the data disclosed herein, particularly genotype data, using any of the tissue samples taught herein. The bulls are then certified as possessing particular genetic traits, particularly the genotype for the ob gene, whether homozygous or heterozygous. The semen obtained from such bulls is also certified as possessing the traits. The genetic data can be associated with other data, such as that disclosed herein, for example, health, feed conditions, growth data, vaccinations and parentage. Steps of the method comprise requesting and obtaining sampling equipment, removing a sample from a bull, sending the sample to a testing site or an entity that provides the sample to a testing site, determining the genotype of the animal, e.g., obtaining the genotype for the ob gene, and entering the data of the genotype in a database. The testing site can be outsourced or performed in house.

The database is accessible to those to whom access has been provided. Access can be provided through rights to access or by subscription to specific portions of the data. For example, the database can be accessed by owners of the animal, the test site, the entity providing the sample to the test site, feedlot personnel, and veterinarians. The data can be provided in any form such as by accessing a website, fax, email, mailed correspondence, automated telephone, or other methods for communication. This data can also be encoded on a portable storage device, such as a microchip, that can be implanted in the animal. Advantageously, information can be read and new information added without removing the microchip from the animal.

The present invention comprises systems for performing the methods disclosed herein. Such systems comprise devices, such as computers, internet connections, servers, and storage devices for data. The present invention also provides for a method of transmitting data comprising transmission of information from such methods herein discussed or steps thereof, e.g., via telecommunication, telephone, video conference, mass communication, e.g., presentation such as a computer presentation (e.g. POWERPOINT), internet, email, documentary communication such as a computer program (e.g. WORD) document and the like.

Systems of the present invention comprise a data collection module, which includes a data collector to collect data from an animal or embryo and transmit the data to a data analysis module, a network interface for receiving data from the data analysis module, and optionally further adapted to combine multiple data from one or more individual animals, and to transmit the data via a network to other sites, or to a storage device.

More particularly, systems of the present invention comprise a data collection module, a data analysis module, a network interface for receiving data from the data analysis module, and optionally further adapted to combine multiple data from one or more individual animals, and to transmit the data via a network to other sites, and/or a storage device. For example, the data collected by the data collection module leads to a determination of the absence or presence of the ob gene in the animal or embryo, and for example, such data is transmitted to a feedstock site when the feeding regimen of the animal is planned.

In another example, cows that are predisposed to mastitis based upon their genetic profiles can be targeted for antibiotic treatment as well as a visit by a veterinarian. In general, cows that are better for milking are more predisposed to mastitis. Instead of treating every dairy cow with antibiotics, the farmer can identify cows predisposed to mastitis (e.g., by identifying the SNP in the CXCR2 gene indicating a predisposition to mastitis) using the methods described herein. Thus, the dairy farmer can optimize the efficiency of treating dairy cattle for mastitis by only treating the dairy cattle genetically predisposed to mastitis as opposed to all of the cows in the herd. Moreover, the dairy farmer can further optimize efficiency by targeting the cows genetically predisposed to mastitis for treatment to a veterinarian. Thus, the farmer can minimize costs by sending only the cows genetically predisposed to mastitis to a veterinarian instead of the entire herd. Furthermore, especially in an embodiment where the data is implanted on a microchip on a particular animal, the farmer can optimize the efficiency of managing the herd because the farmer is able to identify the genetic predispositions of an individual animal as well as past, present and future treatments (e.g., vaccinations and veterinarian visits).

The invention also provides for accessing other databases, e.g., herd data relating to genetic tests and data performed by others, by datalinks to other sites. Therefore, data from other databases can be transmitted to the central database of the present invention via a network interface for receiving data from the data analysis module of the other databases.

The invention relates to a computer system and a computer readable media for compiling data on an animal, the system containing inputted data on that animal, such as but not limited to, ob genotype, DNA testing, thyroglobulin testing, leptin, MMI^{(Meta Morphix Inc}, Bovine spongiform encephalopathy (BSE) diagnosis, brucellosis vaccination, FMD (foot and mouth disease) vaccination, BVD (bovine viral diarrhea) vaccination, SUREBRED pre-conditioning progream, estrus and pregnancy results, tuberculosis, hormone levels, food safety/contamination, somatic cell counts, mastitis occurrence, diagnostic test results, milk protein levels, milk fat, vaccine status, health records, mineral levels, trace mineral levels, herd performance, and the like. The data of the animal can also include prior treatments as well as suggested tailored treatment depending on the genetic predisposition of that animal toward a particular disease. For example, for an animal that is genetically predisposed to mastitis can be targeted for treatment for antibiotics.

A computer readable media may contain such data as described above. The invention also relates to a method of doing business comprising providing to a user the computer system described herein or the media described herein.

The invention provides for a computer-assisted method for predicting which livestock animals possess a physical characteristic comprising: using a computer system, e.g., a programmed computer comprising a processor, a data storage system, an input device and an output device, the steps of: (a) inputting into the programmed computer through the input device data comprising a genotype of an animal, (b) correlating a physical characteristic predicted by the genotype using the processor and the data storage system and (c) outputting to the output device the physical characteristic correlated to the genotype, thereby predicting which livestock animals possess a physical characteristic.

The invention also provides for a computer-assisted method for improving livestock production comprising: using a computer system, e.g., a programmed computer comprising a processor, a data storage system, an input device and an output device, the steps of: (a) inputting into the programmed computer through the input device data comprising a genotype of an animal, (b) correlating a physical characteristic predicted by the genotype using the processor and the data storage system, (c) outputting to the output device the physical characteristic correlated to the genotype and (d) feeding the animal a diet based upon the physical characteristic, thereby improving livestock production.

The invention further provides for a computer-assisted method for optimizing efficiency of feed lots for livestock comprising: using a computer system, e.g., a programmed computer comprising a processor, a data storage system, an input device and an output device, the steps of: (a) inputting into the programmed computer through the input device data comprising a genotype of an animal, (b) correlating a physical characteristic predicted by the genotype using the processor and the data storage system, (c) outputting to the output device the physical characteristic correlated to the genotype and (d) feeding the animal a diet based upon the physical characteristic, thereby optimizing efficiency of feed lots for livestock.

In one advantageous embodiment, the genotype is an ob genotype. In this embodiment, the physical characteristic correlating to a CC genotype is a low propensity to deposit fat, the physical characteristic correlating to a TT genotype is a high propensity to deposit fat and the physical characteristic correlating to a CT genotype is an intermediate propensity to deposit fat.

A “computer system” refers to the hardware means, software means and data storage means used to compile the data of the present invention. The minimum hardware means of computer-based systems of the invention may comprise a central processing unit (CPU), input means, output means, and data storage means. Desirably, a monitor is provided to visualize structure data. The data storage means may be RAM or other means for accessing computer readable media of the invention. Examples of such systems are microcomputer workstations available from Silicon Graphics Incorporated and Sun Microsystems running Unix based, Linux, Windows NT or IBM OS/2 operating systems.

“Computer readable media” refers to any media which can be read and accessed directly by a computer, and includes, but is not limited to: magnetic storage media such as floppy discs, hard storage medium and magnetic tape; optical storage media such as optical discs or CD-ROM; electrical storage media such as RAM and ROM; and hybrids of these categories, such as magnetic/optical media. By providing such computer readable media, the data compiled on a particular animal can be routinely accessed by a user, e.g., a feedlot operator.

The term “data analysis module” is defined herein to include any person or machine, individually or working together, which analyzes the sample and determines the genetic information contained therein. The term may include a person or machine within a laboratory setting.

As used herein, the term “data collection module” refers to any person, object or system obtaining a tissue sample from an animal or embryo. By example and without limitation, the term may define, individually or collectively, the person or machine in physical contact with the animal as the sample is taken, the containers holding the tissue samples, the packaging used for transporting the samples, and the like. Advantageously, the data collector is a person. More advantageously, the data collector is a livestock farmer, a breeder or a veterinarian.

The term “network interface” is defined herein to include any person or computer system capable of accessing data, depositing data, combining data, analyzing data, searching data, transmitting data or storing data. The term is broadly defined to be a person analyzing the data, the electronic hardware and software systems used in the analysis, the databases storing the data analysis, and any storage media capable of storing the data. Non-limiting examples of network interfaces include people, automated laboratory equipment, computers and computer networks, data storage devices such as, but not limited to, disks, hard drives or memory chips.

The invention further comprehends methods of doing business by providing access to such computer readable media and/or computer systems and/or data collected from animals to users; e.g., the media and/or sequence data can be accessible to a user, for instance on a subscription basis, via the Internet or a global communication/computer network; or, the computer system can be available to a user, on a subscription basis.

In one embodiment, the invention provides for a computer system for managing livestock comprising physical characteristics and genotypes corresponding to one or more animals. In another embodiment, the invention provides for computer readable media for managing livestock comprising physical characteristics and genotypes corresponding to one or more animals. The invention further provides methods of doing business for managing livestock comprising providing to a user the computer system and media described above or physical characteristics and genotypes corresponding to one or more animals. In one embodiment, the genotypes include ob genotypes of the animals.

Accordingly, the invention further comprehends methods of transmitting information obtained in any method or step thereof described herein or any information described herein, e.g., via telecommunications, telephone, mass communications, mass media, presentations, internet, email, etc.

Unless otherwise defined, all technical and scientific terms used herein have the same meaning as commonly understood by one of ordinary skill in the art of molecular biology. Although methods and materials similar or equivalent to those described herein can be used in the practice or testing of the present invention, suitable methods and materials are described herein. All publications, patent applications, patents, and other references mentioned herein are incorporated by reference in their entirety. In addition, the materials, methods, and examples are illustrative only and are not intended to be limiting.

### EXAMPLES

### Example 1

In the present example, cattle were grouped according to the ob genotype, indicating a cow's genetic predisposition for laying down fat. Cattle homozygous for the T SNP, hereinafter referred to as TT cattle, are expected to achieve a high fat grade and are considered to be the most genetically predisposed to lay down fat. Cattle least genetically predisposed to lay down fat, CC cattle, are homozygous for the C SNP. It will be advantageous to feed CC cattle so as to achieve a lower fat grade, or a lean grade, rather than feed them longer to achieve the high fat grade. Heterozygous cattle, i.e., CT cattle, can be fed longer to achieve a high fat grade, or fed shorter to achieve a lean grade, depending on considerations such as market prices, price trends, feed costs, and availability of further feeder cattle to bring into the feed lot, and other like external considerations.

Genotype testing of feeder cattle in a typical feedlot situation showed a direct correlation between genotype and fat deposition. The cattle were confined in conventional pens, fed conventional rations, and slaughtered when discerned by conventional means, i.e., visually, to be market ready. The cattle were tested to determine the genotype and were traced to the shipping point to determine the palatability grade achieved. Each pen contained a mix of unsegregated CC, CT, and TT cattle.

Results of the first test (Test1) showed that, of 73 Hereford steers tested for geneotype, 36 were CT, 37 were TT, while none were CC. The 73 cattle were graded when slaughtered: The cattle were graded under a Canadian system of grading beef, wherein AAA corresponds to Prime grade, under U.S. standards. 48.5% of the TT carcasses graded AAA and 19.4% of the CT carcasses graded AAA.

In Test 2, of the 50 Charolais—Angus cross steers tested for genotype, 9 were determined to be CC, 28 were CT, and 13 were TT. When slaughtered, 62% of the TT carcasses graded AAA, 29% of the CT carcasses graded AAA, and 11% of the CC carcasses graded AAA.

In Test 3, 13 Charolais cattle in each of 5 pens, or a total of 65 animals, were tested for genotype. Of the 65 cattle, 29 were CC, 24 were CT, and 12 were TT. There was a high degree of breeding in the 65 cattle. When slaughtered, 58.3% of the TT carcasses graded AAA, 45.5% of the CT carcasses graded AAA, and 38.5% of the CC carcasses graded AAA.

### Example 2

Grouping production animals according to their genotype, in addition to the phenotype grouping already used in feedlot practice allows operators to maximize profits by increasing efficiency in livestock production. A group of CC cattle will have the least propensity to deposit fat, making it more profitable for a producer to slaughter this group earlier in the growth curve, near the start of phase three where the growth curve flattens, since these cattle have the least chance of meeting the fat requirements of the optimum grade. Such a group slaughtered early will have a very high percentage of lean carcasses, and this predictability could itself draw premiums from packers seeking to fill orders requiring lean carcasses. On the other hand, a group of TT cattle will have the most propensity to deposit fat. Therefore, it could be more profitable to keep these cattle on feed longer, since it is predictable that a high percentage would accumulate sufficient intramuscular fat so that the carcass would grade AAA and thus receive a premium price. Likewise, knowing that CT cattle deposit fat at an intermediate rate will allow the feed lot operator to manage this group more efficiently and profitably.

Regardless of the desirability and premium paid for any particular body fat condition at any given time, providing the packer with a more uniform group that is predictably fat or lean will provide the feeder with the opportunity to demand and receive a premium, relative to the less uniform groups of cattle presently available. The packer will be able to buy more of the cattle with a body fat condition that he actually needs, while buying less cattle in total. The packer can thus be much better able to manage his inventory, reducing surplus of carcasses with less desirable body fat conditions that would ordinarily be sold at a reduced price.

The predictability of fat deposition allows the feed lot operator to consider the premiums available for fat or lean carcasses, and tailor his decisions to maximize returns. Where production costs are high, as when feed costs are high, the feedlot operator might profit from slaughtering early. When costs are low, it may be more profitable to slaughter later. The feedlot operator can more accurately predict the particular body fat condition of a group of animals at any given point on the growth curve, and thus more effectively make decisions regarding when to slaughter any particular group.

Feeding rations can also be tailored to more specifically achieve a desired body fat condition for each group by managing production animals' genotype generally, and in particular, the TT/CC/CT genotype.

Among animals of the same species and substantially the same age and weight, where other determinants of growth such as health condition and diet are equivalent, smaller framed animals will reach a stage of maturity, exemplified by the start of the third phase of growth, earlier than larger framed animals. Therefore, substantial leptin effects will be evidenced earlier in such smaller framed animals than in larger framed animals.

Where other determinants of growth such as health condition and diet are equivalent, a group of animals of the same species, sharing substantially the same age, weight, and frame type will attain the stage of maturity exemplified by the start of the third phase of growth at a substantially more uniform time than an otherwise equivalent group of animals, the individual members of which do not share substantially the same frame type. Therefore, where other determinants of growth are equivalent, substantial leptin effects will begin to be evidenced at a more uniform time in animals of a group segregated on the basis of frame type than in animals of a group not so segregated.

### Example 3

A method for extracting DNA from a tissue sample comprises the use of an alkali extraction method employing NaOH. However as will be appreciated by those skilled in the art, KOH could also be used. The alkali typically has a concentration from approximately 150 mM to approximately 200 mM. An advantageous reagent is: Solution A: 200 mM NaOH about 0.5 mM Cresol Red.

No manipulation of the tissue sample prior to extraction is required. An operative in a laboratory enters the identifying code into a computer database. The volume of reagents used in the extraction is determined by the tissue type and the size/volume of the tissue sample. Typically, sample size/volume is determined on a unit volume basis with a typical unit volume being in the range of 25 microlitres to 100 microlitres.

A unit volume of 200 mM NaOH (Solution A) is added to the tissue sample in the container. The container is then heated.

The temperature and period of heating should be sufficient to cause the double-stranded DNA material to revert to single strand form. Accordingly, temperatures in a range of from approximately 95° C. to approximately 99° C. are advantageous and have been found to be particularly effective. The identification cell is typically heated in the aforementioned temperature range for a period of between approximately 15 minutes and approximately 25 minutes.

Heating at a temperature of 97° C. for 20 minutes has been found to be particularly effective. Solution A comprises a dye. The dye is selected to render the sample solution which is usually transparent colored. The dye is selected so as not to react with the extracted DNA nor to interfere with the subsequent genetic analysis. An advantageous dye is cresol red. An advantage of cresol red is that it also acts as a pH indicator to show that the sample pH is in the correct range following mixture of Solution A with a second Solution B.

An advantage of incorporation of cresol red into Solution A is that a color change occurs upon combination of Solutions A and B, i.e. a purple to red color change.

Sample pH is important as many molecular genetic analysis methods demand samples of genetic material having a specified pH. In the advantageous analysis method outlined below a pH of approximately 8 to approximately 9 is advantageous.

The use of the dye in the identification cell facilitates rapid, easy and highly visible handling of samples on a macro scale.

Solution B comprises an acid to reduce the pH of the solution. Solution B typically comprises an acid such as TRIS HCl containing HCl at a concentration from about 150 mm to about 250 mm HCl. An advantageous Solution B comprises: Solution B: 100 mM TRIS HCl pH 8.5 200 mM HCl For analysis purposes, it is desirable that equal proportions of the TRIS HCl solution as compared with the NaOH first solution should be utilized.

A unit volume of Solution B is then added to the container. The final solution therefore has a pH of approximately 8.5 based on TRIS HCl. The samples can be frozen or analyzed immediately.

### Example 4

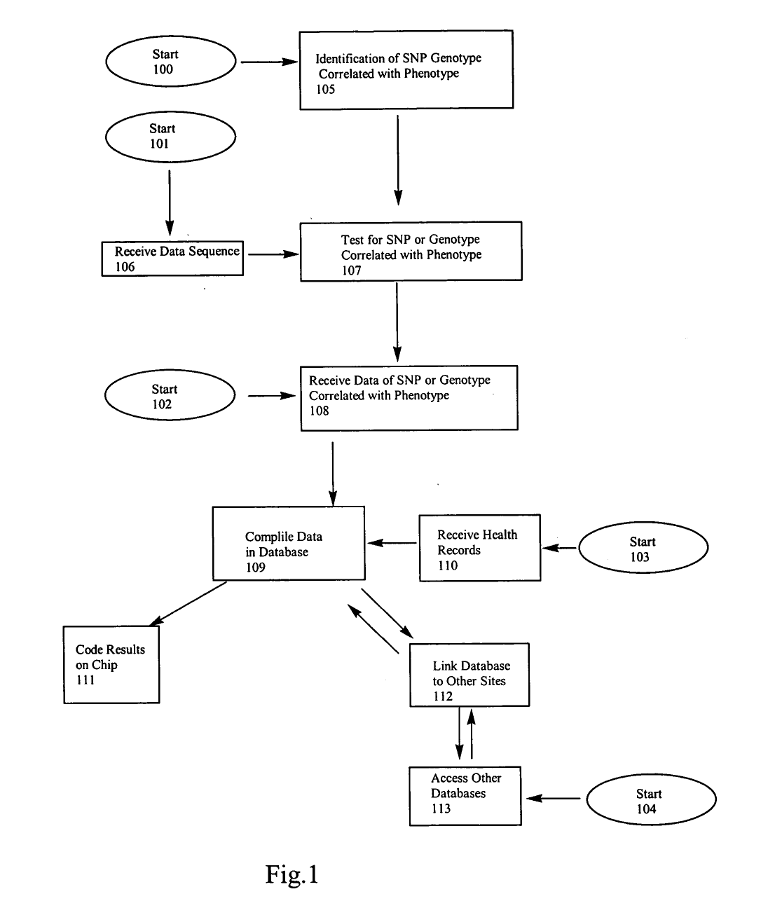

FIG. 1 shows a flowchart illustrating the general overview of input, intermediate steps and output. FIG. 1 demonstrates one method of compiling a database comprising any SNP or genotype correlated with a specific phenotype.

SNPs and/or genotypes that correlate with a specific phenotype can be identified at state 105. Alternatively, samples can be assayed for known SNPs and/or genotypes that correlate with a particular phenotype. For example, but not by limitation, specific genes and/or SNPs that correlate with particular phenotypic characteristics are listed in Table 1. In this instance, the data sequence can be received at state 106. The sequence is advantageously an amplified nucleotide sequence, or alternatively, an amino acid sequence corresponding to the particular gene of interest. The determination of the particular phenotype corresponding to the SNP or genotype in state 107 is ascertainable by the methods described herein (e.g., PCR, LCR, FRET or MALDI-TOF MS). One of skill in the art could adapt these methods into algorithms and into computer programs with routine experimentations for purposes of this invention.

TABLE 1
Correlation of specific genes and/or SNPs with particular phenotypes

| Species | Phenotype | Gene/SNP |
|---|---|---|
| Beef cattle | Carcass quality | Leptin |
| | Food conversion | |
| Beef cattle | Bull fertility predictor | FAA |
| Beef cattle | Meat yield | IGF |
| Beef cattle | Carcass quality/marbling | Thyroglobulin |
| Beef cattle | Feed intake | POMC |
| | Food conversion | |
| Beef cattle | Meat tenderness | Calpastatin/calpain |
| Beef cattle | Feed conversion | UCP2 |
| | Maintenance energy requirements | UCP3 |
| Dairy cattle | Milk yield and content | Leptin |
| Dairy cattle | Milk yield and content | BGHR |
| Dairy cattle | Bull fertility predictor | FAA |
| Dairy cattle | Feed intake | POMC |
| | Food conversion | |
| Dairy cattle | Milk yield and content | DGAT1 |
| Dairy cattle | Milk yield, feed intake | Obese gene |
| Dairy cattle | Mastitis susceptibility | CXCR2 |
| Swine | Meat yield | IGF-2 |
| Swine | Fat deposition | Leptin |

Once data is received correlating the SNP or genotype with a particular phenotype in state 108, either from the test in state 107 or inputted if the data was previously available, the data is compiled in a database in state 109. The database can comprise general data of correlations between SNPs and/or genotype and a particular phenotype. The database can also contain data specific to a particular animal, e.g., the genetic propensity for an animal to deposit fat. Health records of a particular animal (e.g., vaccinations or diagnoses of varying conditions and/or diseases) in state 110 can also be inputted into the database of state 109. In addition, the database of state 109 as it pertains to a specific animal can be encoded on the chip of state 111 for attachment to the specific animal, e.g., on its ear.

In addition, or in the alternative, the database of state 109 can also be linked to other sites (state 110). For example, the database of state 109 can link to other databases (through the link of state 112) and/or provide access to other databases (state 113). Conversely, the other databases of state 113 can link to the database of 109 through the link of state 112.

While the invention has been described with reference to specific methods and embodiments, such description is for illustrative purposes only. The words used are words of description rather than of limitation. It is to be understood that changes and variations may be made by those of ordinary skill in the art without departing from the spirit or scope of the present invention, which is set forth in the following claims.

1. A computer-assisted method for predicting which livestock animals possess a physical characteristic comprising: using a computer system, e.g., a programmed computer comprising a processor, a data storage system, an input device and an output device, the steps of: (a) inputting into the programmed computer through the input device data comprising a genotype of an animal, (b) correlating a physical characteristic predicted by the genotype using the processor and the data storage system and (c) outputting to the output device the physical characteristic correlated to the genotype, thereby predicting which livestock animals possess a physical characteristic.

2. A computer-assisted method for improving livestock production comprising: using a computer system, e.g., a programmed computer comprising a processor, a data storage system, an input device and an output device, the steps of: (a) inputting into the programmed computer through the input device data comprising a genotype of an animal, (b) correlating a physical characteristic predicted by the genotype using the processor and the data storage system, (c) outputting to the output device the physical characteristic correlated to the genotype and (d) feeding the animal a diet based upon the physical characteristic, thereby improving livestock production.

3. A computer-assisted method for optimizing efficiency of feed lots for livestock comprising: using a computer system, e.g., a programmed computer comprising a processor, a data storage system, an input device and an output device, the steps of: (a) inputting into the programmed computer through the input device data comprising a genotype of an animal, (b) correlating a physical characteristic predicted by the genotype using the processor and the data storage system, (c) outputting to the output device the physical characteristic correlated to the genotype and (d) feeding the animal a diet based upon the physical characteristic, thereby optimizing efficiency of feed lots for livestock.

4. The methods of claims 2-3 wherein the genotype is an ob genotype.

5. The method of claim 4 wherein the physical characteristic correlating to a CC genotype is a low propensity to deposit fat.

6. The method of claim 4 wherein the physical characteristic correlating to a TT genotype is a high propensity to deposit fat.

7. The method of claim 4 wherein the physical characteristic correlating to a CT genotype is an intermediate propensity to deposit fat.

8. A method of transmitting data comprising transmission of information from such methods herein discussed or steps thereof, e.g., via telecommunication, telephone, video conference, mass communication, e.g., presentation such as a computer presentation (e.g. POWERPOINT), internet, email, documentary communication such as a computer program (e.g. WORD) document and the like.

9. A computer system for managing livestock comprising physical characteristics and genotypes corresponding to one or more animals.

10. A computer readable media for managing livestock comprising physical characteristics and genotypes corresponding to one or more animals.

11. A method of doing business for managing livestock comprising providing to a user the computer system of claim 9 or the media of claim 10 or physical characteristics and genotypes corresponding to one or more animals.

12. The genotypes of claims 9-11 wherein a genotype is an ob genotype.

13. A computer-assisted method for optimizing efficiency of treating mastitis in dairy cattle comprising: using a computer system, e.g., a programmed computer comprising a processor, a data storage system, an input device and an output device, the steps of: (a) inputting into the programmed computer through the input device data comprising a genotype of an animal, (b) correlating a physical characteristic predicted by the genotype using the processor and the data storage system, (c) outputting to the output device the physical characteristic correlated to the genotype and (d) treating the animal based upon the physical characteristic, thereby optimizing efficiency of treating mastitis in dairy cattle.

14. The method of claim 13 wherein the genotype is CXCR2.

15. The method of claim 14 wherein the physical characteristic correlates to developing mastitis.

16. The method of claim 14 wherein treating the animal comprises administration of antibiotics.

17. The method of claim 16 wherein treating further comprises a visit to a veterinarian to prevent mastitis.
